(19) United States
(12) Patent Application Publication
(10) Pub. No.: US 2025/0325256 A1
(43) Pub. Date: Oct. 23, 2025

(54) LIPID PATCH

(71) Applicant: SENSACH AB, LUND (SE)

(72) Inventors: Johan ENGBLOM, LUND (SE); Sebastian BJÖRKLUND, MALMÖ (SE); Maxim MORIN, TRELLEBORG (SE); Skaidre JANKOVSKAJA, MALMÖ (SE); Tautgirdas RUZGAS, BJÄRRED (SE)

(21) Appl. No.: 18/701,777

(22) PCT Filed: Oct. 19, 2022
(86) PCT No.: PCT/EP2022/079083

§ 371 Date: Apr. 16, 2024

(30) Foreign Application Priority Data
Oct. 19, 2021 (SE) 2151273-6

(51) Int. Cl. A61B 10/00; A61B 5/00

(52) U.S. Cl. CPC A61B 10/0035; A61B 5/411

(57) ABSTRACT

The present invention related to a matrix, a patch and a method for non-invasive sampling of at least one endogenous substance on a skin surface of an individual, wherein the matrix comprises at least one amphiphile, wherein the amphiphile, alone or in combination with at least one structurally related amphiphile, forms a non-lamellar liquid crystalline phase together with an aqueous polar solvent mixture, said matrix comprising a water activity of at least 0.85 in a temperature range of 20-40° C., wherein the matrix is configured to extract said at least one endogenous substance on the skin surface of the individual.

### FIELD OF THE INVENTION

The present invention relates to a matrix and a method for non-invasive sampling of at least one endogenous substance on the skin surface of an individual. Further, the present invention relates to a patch comprising said matrix and a use for non-invasive sampling of at least one endogenous substance by extracting said at least one endogenous substance on an area of the skin of an individual.

### TECHNICAL BACKGROUND

Many skin disorders may be identified simply by visually revealing abnormal skin characteristics. However, the clinical diagnosis of certain inflammatory skin diseases or skin disorders such as psoriasis and skin cancer rely on tissue biopsy and staining. The decision to take a biopsy test is usually postponed until visual inspection indicates high probability of the disease. The costs for taking biopsies more frequently cannot be justified since skin inflammation and cancer affect a tremendous amount of people. Psoriasis for example, which is a chronic inflammatory disease, affects about 2% of world's population. Furthermore, occurrence of non-melanoma and melanoma skin cancers increases rapidly and today one out of three cancer diagnoses is a skin cancer. Worldwide 2 to 3 million non-melanoma and 132 000 melanoma (doubled over the last decade) cases are registered each year. At the same time, links between inflammation and cancers becomes clearer. It is now established that persistent inflammations contribute and may lead to cancer development. Therefore, detection of endogenous substances associated with inflammation by non-invasive, i.e. biopsy less, methods in suspected cancer lesions would give an early warning on cancer onset. If paralleled with biopsy less detection of endogenous substances associated with cancer such tools would be diagnostic and, thus, very desirable. In fact, most skin cancers are curable if detected early enough.

Due to the high potential of non-invasive methods of analysis, fundamental research activities within this field has increased significantly over the last 15 years. One limiting factor in the development of non-invasive devices for detection of skin disorders or skin diseases has so far been the limited access to endogenous substances associated with skin diseases. Biopsies are necessary today because the diagnostic decision is based on analysis of high molecular weight (HMW) endogenous substances, e.g. cytokines or cell surface receptors. Since HMW endogenous substances do not leak to the skin surface, these endogenous substances cannot be used for non-invasive detection.

Thus, there is a need for improved methods and devices that does not require a biopsy and minimizes the need for clinical laboratory expertise.

### SUMMARY OF THE INVENTION

In view of the above, it is an object of the present invention to provide a cost-efficient, point-of-care device, such as a patch, that does not require a biopsy and minimizes the need for clinical laboratory expertise.

Although skin is a tough biological barrier, it allows diffusion of low molecular weight (LMW) compounds, i.e. <500 Da. The “500 Da rule” is a well-established rule of thumb and therefore larger molecules may very well penetrate if conditions are favorable. With favorable conditions means e.g. hydrated skin, i.e. a lower water activity gradient over skin, and/or increased skin temperature, providing higher mobility to the skin barrier constituents and thereby higher permeation to hydrophilic and lipohilic compounds, and any compounds in between. Hence, the object of the present invention is to provide a matrix which when attached to or when in close proximity to a skin surface of an individual, is designed to extract any endogenous substance on the skin surface of an individual, such as substances with a MW<2000 Da, <1000 Da, <500 Da. Thus, another object is to achieve robust non-invasive or minimally invasive monitoring of LMW endogenous substances of skin disorders or skin diseases, in particular LMW endogenous substances of inflammation and cancer.

Another object is to provide a matrix which is designed for non-invasive sampling of both hydrophilic and hydrophobic endogenous substances on the skin surface of an individual. By “sampling” means gathering of matter from the body, e.g. endogenous substances, to aid in the process of a medical diagnosis and/or evaluation of an indication for treatment, further medical tests or other procedures. Thus, an effect of the non-invasive sampling of the present invention is to determine if the individual is affected by a disease, such as an inflammatory disease.

Another object is to provide a matrix which is designed to both being able to deliver a substance to the skin of an individual and subsequently extracting endogenous substances from the surface of the skin in order to determine if the individual is affected by a disease, such as an inflammatory disease. Hence the objective of the present invention is to detect the triggered endogenous response from delivering a substance from the matrix, by delivering either i) a drug treating a disease and the corresponding progress in “healing”—though sometimes a long term thing, where treatment over time and monitoring over time overlaps; ii) an irritant or an allergen triggering a more immediate response; or iii) a substance for tox testing of said substance.

Another object is to provide a matrix which is useful for the determination if an irritant or allergen will stimulate a response in an individual.

Another object is to provide a matrix which is useful for the determination if a substance delivered to the skin of an individual is toxic to the individual.

Another object is to provide a matrix which is useful for the determination of the hydration level of the skin. The matrix is thus also useful in determining the efficacy of moisturizers on the skin. This may be established by using swelling theory derived by Engblom J and Hyde ST; On the Swelling of Bicontinuous Lyotropic Mesophases; J. Phys II (France), 5 (1995) 171-190.

One further objective is to provide a method of extracting endogenous substances or analytes from the patch of the invention after it has been removed from the skin, by adding salt and thereby screen charges and “deswell the system”. From this, phase separation may occur, and free water may easily be sampled for further analysis by any method, such as HPLC (high performance liquid chromatography), UPLC (ultra performance liquid chromatography), LC-MS (liquid chromatography-mass spectrometry), LC-MS/MS (liquid chromatography-tandem mass spectrometry), GC-MS (gas chromatography-mass spectrometry), NMR (nuclear magnetic resonance) etc.

To achieve at least one of the above objects and other objects that will be evident from the following description, a matrix having the features defined in claim 1 is provided according to the present inventive concept. A patch comprising the matrix is provided according to claim 7. A method for sampling is provided according to claim 8 and a use of the matrix is provided according to claim 13. Preferred variations to the inventive concept will be evident from the dependent claims.

According to a first aspect there is provided a matrix for non-invasive sampling of at least one endogenous substance on a skin surface of an individual, wherein the matrix comprises at least one amphiphile, wherein the amphiphile, alone or in combination with at least one structurally related amphiphile, forms a non-lamellar liquid crystalline phase together with an aqueous polar solvent mixture, said matrix comprising a water activity of at least 0.85 in a temperature range of 20-40° C. The matrix is configured to extract said at least one endogenous substance on the skin surface of the individual.

With the term “Endogenous substance” means a substance that originate from within a biological system such as an organism, tissue, or cell. Endogenous substances contrast with exogenous ones, such as drugs, which originate from outside of the organism. The at least one endogenous substance may be associated with inflammatory diseases. The at least one endogenous substance may be associated with cancer. The at least one endogenous substance may be associated with skin diseases or skin disorders. Inflammatory diseases may be selected from the group consisting of: seborrheic dermatitis, rosacea, lupus, psoriasis, eczema, ichtyosis, skin cancer, diabetes and inflamatory bowel disease. Skin cancer may be selected from the group consisting of basal cell carcinoma, squamos cell carcinoma and melanoma. Although diabetes and inflammatory bowel disease are not to be considered as actual disorders of the skin, there are evidence or postulates asserts that systemic diseases are reflected in the skin and maybe thus in biomarker composition extracted from the skin. Inflammatory systemic diseases may usually be enforced by bad and/or leaky skin barrier. At the same time, systemic mediators of inflammation that opens e.g. tight junctions in intestine, will open also tight junctions in skin.

Endogenous substances, such as various endogenous amino acids, lipids, etc, and their related substances, associated with said inflammatory diseases are usually prone to be emitted through the skin of an individual. The at least one endogenous substance may be a low molecular weight substance with a molecular weight of typically up to 2000 Da, typically up to 1000 Da, typically up to 500 Da. In one embodiment the at least one endogenous substance has a molecular weight of at most 1000 Da. In another embodiment the at least one endogenous substance has a molecular weight of at most 500 Da. There is no upper limit in molecular weight of the substances that the matrix may extract, however most endogenous substances that may diffuse through skin is below 500 Da.

The at least one endogenous substance may range from hydrophilic compounds through amphiphilic compounds to hydrophobic compounds. The at least one endogenous substance may be selected from hydrophilic compounds and hydrophobic compounds. The at least one endogenous substance may be extracted with other endogenous substances or its metabolites or a mixture thereof. The at least one endogenous substance may be selected from amino acids and metabolites of amino acids or mixtures thereof. Examples of amino acids or metabolites thereof may be Tyrosine, Phenylalanine, Trypthophan, Kynurenine, Alanine, Aminomalonate (Malonic acid), Asparagine, Aspartic acid, Citric acid, Citrulline, Glucose, Glutamic acid, Glutamine, Glyceric acid, Glycerol, Glycine, Glycolic acid, Isoleucine, Lactic acid, Leucine, Lysine, Malate (Malic acid), Oleic acid, Ornithine, Oxalic acid, Oxoproline (Pyroglutamic acid), Proline, Serine, Succinic acid, Sucrose, Threonine, Urea, Urocanic acid, Valine, ½-monoolein, ½-monopalmitin, 1-monostearin, 2-monostearin, Palmitic acid, Pelargonic acid or Stearic acid. Preferably the amino acids or metabolites thereof may be selected from the group consisting of: tyrosine (Tyr), phenylalanine (Phe), trypthophan (Trp) and kynurenine (Kyn) or related substances such as kynurenic acid, which may also be abbreviated Kyn. This may be referenced to the Kynurenine pathway, wherein Tryptophan converts Kynurenine, which then converts into Kynurenic acid or other related substances via 3-Hydroxykynurenine.

With the term “amphiphile” means a molecule that is possessing both a hydrophilic group, i.e. having water-loving or polar properties, and a lipophilic group, i.e. having fat-loving properties. The lipophilic group may be a large hydrocarbon chain, such as a long chain of the form CH₃(CH₂)ₙ, wherein n may be in the range of 4-30, preferably n may be in the range of 8-24, preferably n may be in the range of 12-22. Such hydrocarbon chain may also comprise one or several carbon-carbon double and/or triple bounds. The hydrophilic group may either be a charged group or a polar uncharged or non-ionic group. The charged groups may be either anionic, such as carboxylates, sulfates, sulfonates or phosphates, or cationic, such as ammonium or positively charged amines. For example, primary, secondary and tertiary amines can be positively charged depending on pH. Further, quaternary ammonium compounds are an important class of zwitterionic surfactants. As a comparison may classical commercial nonionic surfactants comprise for example poly(alkaline oxide)block co-polymers, oligomeric alkyl-ethylene oxides, alkyl-phenol polyethylenes, or sorbitan esters. Examples of polar uncharged or non-ionic groups are alcohols or thiols. The amphiphile may have at least one lipophilic part and at least one hydrophilic part. As a result of having both lipophilic and hydrophilic parts, the matrix comprising the at least one amphiphile may dissolve in water and to some extent in non-polar organic solvents. With the term “structurally related amphiphile” means a molecule that may intercalate into a lipid monolayer or bilayer to form a homogenous liquid crystal together with the “parent molecule”, such as the at least one amphiphile, and a polar solvent (like water). Such a molecule could have a similar hydrocarbon chain length as the “parent molecule”+/−4 carbons and preferably +/−2 carbons, with an optional headgroup at the end of one or both ends of the hydrocarbon chain, and while the polar headgroup may be any optional type, such as the charged or polar uncharged or non-ionic groups presented above. By combining different amphiphiles a more favorable “average critical packing parameter” may be obtained i.e. that gives non-lamellar structures, even if each amphiphile individually are not able to form non-lamellar structures. The “average critical packing parameter” or “CPP” may be described as when CPP is equal to one (CPP=1) this gives a lamellar phase. However, if the CPP is slightly higher than 1 this may give a “reversed” type non-lamellar bicontinuous cubic phase. Moreover, with further increase in CPP the average shape of the amphiphile will more resemble an inverted or truncated cone or wedge, and thus generate e.g. a reversed hexagonal phase, ultimately reversed micelles. The same symmetrical behavior may be seen with a CPP below 1, wherein “normal” phases are formed, ultimately the CPP for normal spherical micelles may be smaller than ⅓ (CPP<⅓). Thus, the preferred CPP for the non-lamellar liquid crystalline phase of the present invention may be in the range of ⅓ to 3 (⅓<CPP<3), preferably the CPP may be in the range of ½ to 2 (½<CPP<2).

Non-lamellar liquid crystals or liquid crystalline phases may be identified with either of a number of methods, preferably with two independent determinants as best practice, such as:
visual inspection, which may be based on certain characteristics of alternative phases, such as visual appearance, birefringence as seen when viewed between crossed polars, viscosity, ringing gel, i.e. a characteristic for cubic phases, etc.;
polarized light optical microscopy (PLOM);
small and wide angle X-ray scattering/diffraction (SWAXS/SWAXD), wherein either scattering or diffraction preferably, since the methods more or less refers to the same method and the choice of referring to the method may largely depend on the background of the scientist performing the experiment; or
nuclear magnetic resonance (NMR) spectroscopy.

With “crossed polars” means “crossed polarized windows”, c.f. polarized sun glasses. Thus, when two perpendicular oriented polarized windows are used, no light passes through. This is a test of the phase itself being anisotropic, e.g. lamellar or hexagonal phase, then turning the light to allow it to pass through the two polarized windows. In the opposite case, an isotropic phase, e.g. cubic phases, does not perturb light.

Further, SAXS/SAXD relates what type of liquid crystal or crystal, or what type of dispersed particles that may be present in a sample, while WAXS/WAXD relates whether the sample is liquid crystalline or crystalline and also what type of crystal or crystals it comprises, i.e. how the hydrocarbon chains are organized in the unit cell.

The water activity (aw) is a thermodynamic measure of water expressed as the vapor pressure of water in a sample divided by vapor pressure of pure water at a given temperature.

There are three basic water activity measurement systems. The water activity may for example be measured by Resistive Electrolytic Hygrometers (REH), Capacitance Hygrometers, and Dew Point Hygrometers (sometimes called chilled mirror). The typical water activity measurement system uses a sealed, temperature-controlled chamber. A sample is placed in the chamber and sealed. The free water is allowed to escape into the air in the chamber to eventually reach a condition of equilibrium. The water activity of the matrix of the present invention may be measured according to the method disclosed in Bjorklund S & Kocherbitov V, Langmuir 32 (2016) 5223-5232. The aw of aqueous surfactant solutions was measured with a NovaSina LabMaster-aw apparatus at 25° C. The instrument was calibrated with saturated salt solutions (standards provided with the instrument) at suitable relative humidity (RH) before measurements. At equilibrium the water activity of the air in the chamber is measured. The range of water activity is from 0.0 to 1.0 and it compares linearly to relative humidity (% RH) in air as aw 0=0% RH, and aw 1=100% RH. Water activity is for example a critical factor in determining quality and safety of foods. It affects shelf life, safety, texture, flavor, and smell. For example, bacteria do not grow at water activities below aw 0.90 and most molds cease to grow at water activities below a)w 0.80. Thus, for example, the water content of the matrix according to the present invention may be at least 20 wt %. However, the matrix according to the present invention comprises a water activity of at least 0.85 in a temperature range of 20-40° C. Preferably, the water activity is of at least 0.9 in a temperature range of 20-40° C. The higher water activity is crucial for good skin permeability as this may differ by an order of magnitude between dry and fully hydrated skin. This, means that a matrix or a patch according to the present invention with high water activity could be e.g. either a hydrogel (1% thickener, such as a polymer, and water) or a lipid based vehicle, i.e. the amphiphile together with a polar solvent like water (which is needed for the amphiphile to be able to self assembly into the desired structures), alone, or in combination with at least one structurally related amphiphile according to the present invention. Hydrogels may be described as polymeric network structures able to imbibe large amount of water. According to the present invention both natural and synthetic polymers may be used. Examples of natural polymers are polypeptide hydrogels (e.g. gelatin and derivatives), polysaccharide hydrogels (e.g. chitosan, dextran, hyaluronan, starch, cellulose and derivatives), whereas synthetic hydrogels may be constituted using e.g. polyesters.

By applying an occlusive patch, comprising the matrix of the invention with high water activity on skin it will hydrate the skin and thus increase the mobility of its barrier constituents and in turn the permeability of skin to both hydrophilic and lipophilic substances. The liquid crystalline phase can be designed so that it sustains a high water activity of the patch matrix, i.e. of at least 0.85, and does not undergo structural phase transitions during application. For example, the present invention may show real data for area vs water content of close to 500 m²/cm³ at 30 wt % water. The present invention provides a matrix with both hydrophilic and lipophilic domains together with an extensive interfacial area between the two, which may be up to the size of two tennis courts per mL, i.e. the matrix may have an interfacial area up to 520 m²/mL. In addition the matrix may have a high water activity, i.e. of at least 0.85, that hydrates the tissue and facilitates solute extraction when a patch according to the present invention is applied on the skin.

In one embodiment there is provided a matrix for non-invasive sampling of at least one endogenous substance on a skin surface of an individual, wherein the matrix comprises at least one amphiphile, wherein the amphiphile, alone or in combination with at least one structurally related amphiphile, forms a non-lamellar liquid crystalline phase together with an aqueous polar solvent mixture.

The aqueous polar solvent mixture of the matrix according to the present invention may comprise water or water in combination with a polar co-solvent. The polar co-solvent may be a protic polar co-solvent or an aprotic polar co-solvent. The protic polar co-solvent may be selected from linear or branched C₁-C₄ alkyl alcohol, such as ethanol, glycerol, isopropyl alcohol, t-butanol, propanol, glycerol; acetic acid; and ammonia. The aprotic polar co-solvent may be selected from acetone, tetrahydrofuran (THF) and dimethyl sulphoxide (DMSO). The aqueous polar solvent mixture may comprise water as only solvent. The aqueous polar solvent mixture may comprise water in combination with a polar co-solvent. The polar co-solvent may be selected from the group consisting of: ethanol, glycerol, isopropyl alcohol, t-butanol, propanol, glycerol, acetic acid, ammonia, acetone, tetrahydrofuran (THF) and dimethyl sulphoxide (DMSO). The ratio of water to polar co-solvent in the aqueous polar solvent mixture may be in the range of 100:0 to 10:90.

The matrix according to the present invention has the advantages that it improves transport of substances through the skin of an individual. This is by hydration of the skin and thus facilitating partition and extraction of substances, wherein the high water activity is the key. The inclusion of other polar solvents may also enhance permeability to various solutes by affecting the skin barrier. Favorable partitioning requires an optimal matrix for extraction, thus the present invention provides with a matrix comprising liquid crystals with their diverse phase behavior in combination with water, alone or in combination with polar co-solvents, offers this interesting opportunities.

Another advantage with the matrix of the present invention is that the matrix may be designed to comprise a certain polar solvent mixture, including the co-solvents of the present invention, depending on the substances that are to be extracted from the surface of the skin of an individual. The matrix may also be designed to comprise water channels of certain dimensions, such as average radius of curvature and channel length per unit cell. A further advantage of this matrix is that the interfacial area between amphiphile and polar solvent mixture per unit cell (given in either interfacial area per unit cell or per unit volume), or for that matter per unit volume, may be tuned and easily span from 100 to 500 m²/cm³. Thus, although both hydrophilic and hydrophobic molecules may be extracted at the same time, the rate of extraction or specificity of the extracted molecule may be directed towards either more hydrophilic molecules or hydrophobic molecules depending on which type of molecules are of interest.

The non-lamellar liquid crystalline phase according to the present invention may be selected from the group consisting of: cubic phase, hexagonal phase, micellar, sponge phase and any intermediate between these or mixtures thereof. Preferably, the non-lamellar phases may be selected from cubic phases and hexagonal phases or a mixture thereof.

Both the cubic phases and the hexagonal phases are semisolid “gel-like” structures with high internal interfacial area between amphiphile and polar solvent mixture. Bicontinuous cubic phases furthermore comprise an extensive interconnected network of polar and apolar domains, which together with the high internal interfacial area may accommodate any type of molecule to be extracted from the skin, being hydrophilic, amphiphilic or lipophilic. Similar any type of molecule to be administered to the skin from the patch may be accommodated in such a matrix.

The non-lamellar phase may be of a reversed type. The non-lamellar phase may be a bicontinuous phase. The non-lamellar phase may be a bicontinuous cubic liquid crystalline phase. The non-lamellar phase may be a bicontinuous cubic liquid crystalline phase within a range in interfacial area per mL of at least 100-500 m²/cm³. The non-lamellar phase may be a hexagonal liquid crystalline phase. The non-lamellar phase may be a hexagonal liquid crystalline phase within a range in interfacial area per mL of at least 50-300 m²/cm³.

The amphiphile according to the present invention may be selected from the group consisting of: natural lipids, synthetic lipids, ionic lipids and surfactants. Natural lipids may be selected from the group consisting of insoluble swelling amphiphilic lipids: acylglycerols, glycerol ethers, phospholipids, glycosphingolipids, glycosylglycerides, acid-soaps, alfa-hydroxy fatty acids. Acylglycerols may be selected from the group consisting of saturated monoglycerides, unsaturated monoglycerides, unsaturated diglycerides, mono- or poly-unsaturated monoglycerides, mono- or poly-unsaturated diglycerides: such as glyceryl monolaurate, glyceryl monomyristate, glyceryl monooleate, glyceryl monoelaidate, glyceryl monoeuricin, glyceryl monolinoleate, glyceryl dioleate, and phytantriol. Unsaturated monoglycerides may comprise oleic acid as the apolar part. The amphiphile may be monoolein (GMO), monoelaidin (GME), monolinolein (MLO) or phytantriol (PHYT) in combination of each other or in combination with an oil e.g. a triglyceride oil (such as medium chain triglycerides (MCT)), or a free fatty acid (such as oleic acid). The amphiphile may be monoolein (GMO), monoelaidin (GME), monolinolein (MLO) or phytantriol (PHYT) in combination of each other or in combination with a structurally related amphiphile being an acyl glycerols, a glycerol ether, a phopholipids. Glycerol ethers may be selected from the group comprising alkyl ethers, alk-1-enyl ethers and betaine lipids, such as monoalkyl ethers (e.g., batyl alcohol, chimyl alcohol), dialkyl ethers (e.g., diphytanyl glycerol), monoalk-1-enyl ethers, dialk-1-enyl ethers; and betaine lipids such as DGTS, DGTA. Phospholipids, comprising glycerophospholipids (such as dioleyl phosphatidylglycerol, dioleyl diphosphatidylglycerol (cardiolipin), dioleyl phosphatidylserine dioleyl phosphatidic acid, dioleyl phosphatidylethanolamine, dioleyl phosphatidylcholine), and sphingophospholipids (such as sphingomyelin), may come as diacylesters, plasmalogens, monoacyl monoethers, diethers, monoacyl (lyso) forms and phosphono forms. Natural lipids may further be selected from the group consisting of non-ionic, anionic, zwitterionic and cationic lipids. The amphiphile may be selected from the group consisting of: glyceryl monooleate, glyceryl monoelaidate, glyceryl monolinoleate, glyceryl dioleate, dioleyl phosphatidylglycerol, distearyl phosphatidylglycerol, dioleyl phosphatidyl ethanolamine, dioleyl phosphatidylcholine and phytantriol or mixtures thereof. The amphiphile according to the present invention may be an acyl glycerol or a glycerol ether in combination with an anionic (e.g., distearoyl phosphatidylglycerol), zwitterionic (e.g., dioleyl phosphatidylethanolamine) or cationic (e.g., dioleoyl-3-trimethylammonium propane) lipid. Synthetic lipids may be identical to their natural analogs. Examples of synthetic lipids may be technical grade diglycerol mono-isostearate surfactants, e.g. C41V (from the Nisshin OilliO Group Ltd., Japan). The surfactant according to the present invention may be selected from the group forming non-lamellar liquid crystals together with polar solvents, as exemplified by didodecyl diamonium bromide (DDAB) or dodecaoxyethylene mono-n-dodecyl ether (C12EO12)/water.

By combing at least one lipid amphiphile with at least one structurally related amphiphile a favourable average critical packing parameter (CPP) may be achieved, which thus renders non-lamellar structures eventhough each of the amphiphiles on their own are not able to form such a structure.

The amphiphile may be selected from the group consisting of: glyceryl monooleate (GMO), glyceryl monolinoleate, glyceryl dioleate (GDO), dioleyl phosphatidyl ethanolamine (DOPE), dioleyl phosphatidylcholine (DOPC) and phytantriol (PHYT) or mixtures thereof. Preferably the at least one lipid amphiphile is glyceryl monooleate. For example, glyceryl monooleate (GMO) may per definition have the lipid number C18:1. With the term “lipid number” means that fatty acid chains may be described by their lipid numbers on the form CX:D, wherein X is the number of carbon atoms (C) in the fatty acid and D is the number of double bonds in the fatty acid. However, in reality GMO may often be of biological origin. Thus, the distribution in hydrocarbon chain lengths and the degree of saturation, may include 1 or 2 double bonds in the chain. The degree of fatty acid esterification to the glycerol and the positions of the ester links may also vary, thus including some diglycerides, like diolein. The advantage of using glyceryl monooleate in the matrix of the present invention is that it forms bicontinuous cubic structures by itself in combination with a polar solvent, it is well characterized, and it is generally recognized as safe (GRAS). Preferably the at least one lipid amphiphile may also be phytantriol. The advantage of using phytantriol in the matrix of the present invention is that it is more chemically stable, while possessing similar properties as glyceryl monooleate.

The ionic lipids may be selected from the group consisting of: an anionic lipid, a cationic lipid and a zwitterionic lipid.

A further object of the present invention is that the matrix, i.e the amphiphile-polar solvent mixture interface, may be doped with cationic or anionic compounds, to further facilitate extraction of endogeneous charged compounds by electrostatic interactions. Thus, the doping provides tuning properties of the matrix towards extracting components of a specific charge, either negatively, zwitterionic or positively charged. It has been shown that electrostatics may be more potent than interfacial area. Thus, anionic or cationic lipids may provide a strong asset to the matrix. Further, salts may be used to screen electrostatics, by the addition to a matrix and further decompose a liquid crystal comprising charged lipids. Thus, water is expelled from the lipid structure that the may be easy to sample for subsequent analysis by e.g. UPLC, HPLC, LC-MS etc.

The cationic lipid may be selected from the group here represented by dioleoyl-3-trimethylammonium propane (DOTAP), and their mixtures. Preferably the cationic lipid is dioleoyl-3-trimethylammonium propane. The anionic lipid may be selected from the group consisting of dialkyl-phosphatidylglycerol, diphosphatidylglycerol (cardiolipin), phosphatidylserine, phosphatidic acid, phosphatidylethanolamine and phosphatidylcholine, or mixtures thereof. Preferably the anionic lipid is distearyl phosphatidylglycerol. The zwitterionic lipid may be selected from the group consisting of: dialkyl phosphatidylethanolamine, dialkyl phosphatidylcholine, betaine lipids (such as DGTS, DGTA), or mixtures thereof. Preferably the zwitterionic lipid is dioleyl phosphatidylethanolamine. The cationic lipids may be selected from the group consisting of: dioleoyl-3-trimethylammonium propane, and dipalmitoyl-3-trimethylammonium propane, dipalmityl-3-trimethylammonium propane, distearyl-3-trimethylammonium propane and dielaidyl-3-trimethylammonium propane or mixtures thereof. Preferably the cationic lipid may be dioleoyl-3-trimethylammonium propane. An advantage of adding an ionic liquid to the matrix may be that the internal structure of the matrix may be finely tuned by introducing molecules that may suitably alter the critical packing parameter to favour a non-lamellar liquid crystalline phase.

The ratio of the amphiphile and the ionic lipid in the matrix may be in a range from 80:20 to 99.5:0.5 (% w/w), 85:15 to 97.5 (% w/w) or 85:15 to 95:5 (% w/w).

The matrix may further comprise at least one additive selected from the group consisting of: humectant, drug, bioactive agent, irritant and allergen.

The matrix may further comprise a humectant. In one embodiment the matrix may further comprise a drug or a bioactive agent. In another embodiment the matrix may further comprise an irritant or an allergen. The matrix may have a surface area in the range of 0.5-5 cm², preferably 1-3 cm². The matrix may have thickness of 0.1-2 mm, preferably 0.5-1 mm. The matrix may have volume in a range from 50-500 μl, preferably 100-300 μl. With “drug” means for example Nonsteroidal Anti-Inflammatory Drugs (e.g., diclofenac, nepafenac, ketorolac, indomethacin, ketoprofen, piroxicam, flurbiprofen, tenoxicam, naprofen, ibuprofen, felbinac; topical steroid drug e.g. Corticosteroid drugs including cortisone, hydrocortisone, prednisone fluorometholone, mometasone, betametasone. With “bioactive agent” means that it could in principle be any humectant, allergen or irritant. A “bioactive agent” could also be e.g., plant and insect-originated bioactive molecules for pharmaceutical applications such as novel anti-cancer, anti-inflammatory, anti-microbial, and anti-diabetic agents. With “humectant” means a hydrophilic substance used to keep things moist. An “allergen” is a substance that causes an allergic reaction. Most allergens are proteins. There are also small molecules that can bind to antibodies but do not themselves trigger an allergic reaction, but become immunogenic if they bind to a protein.

According to a second aspect there is provided a patch comprising a matrix according to the first aspect of the invention.

According to a third aspect there is provided a non-invasive method for sampling of at least one endogenous substance on the skin surface of an individual, the method comprising:
i. placing a matrix according to the first aspect of the invention or a patch according to the second aspect of the invention against an area of the skin surface of an individual;
ii. extracting at least one endogenous substance on the area of the skin surface of the individual; and
iii. determining the presence of said at least one endogenous substance.

The determining step iii may comprise quantifying the at least one endogenous substance by determining a ratio between at least two extracted endogenous substances.

“Endogenous substances” and “endogenous processes” are those that originate from within a system such as an organism, tissue, or cell. Endogenous substances and processes contrast with exogenous ones, such as drugs, which originate from outside of the organism.

An advantage of the present method may be that it is non-invasive or minimally invasive compared to existing methods for assessing for example a disease associated with inflammation which usually involves biopsy sampling. With the term “non-invasive” means a method not requiring the introduction of instruments below the skin or into the body of a human or an animal. With the term “minimally invasive” means e.g. the use of microneedles onto the skin. Thus, with non-invasive method means a procedure not requiring the introduction of instruments into the human or animal body.

The method may further comprise a step of delivering a drug or a bioactive agent prior to and/or simultaneously to step ii from the matrix, to trigger a response that reflects the presence of the at least one extracted endogenous substance. Further, the at least one endogenous substance may be extracted with other endogenous substances or metabolites thereof and the determining step iii comprises estimating a ratio between two of the extracted endogenous substances or metabolites. An advantage of this may be that the response and progression of e.g healing may be monitored in parallel to, or following the treatment.

In another embodiment, the method may further comprise a step of delivering a drug or a bioactive agent prior to and/or simultaneously to step ii from the matrix, to trigger a response that reflects the presence of the at least one extracted endogenous substance, and thus it may be used to estimate if the individual is affected by an inflammatory disease.

The method further comprises a step of delivering an irritant or an allergen prior to and/or simultaneously to step ii from the matrix, to estimate if said irritant or allergen trigger a response in the skin of the individual. An advantage of this may be that the type of response may be more accurately identified and concluded beyond what just visual judgement may convey.

This is because specific endogenous substances or biomarkers may be associated with an allergic or irritating reaction and thus may help the medical doctor to make a diagnosis, compared to the visual inspection for redness of the skin, which is the standard practice today.

The extracting step ii may comprise the use of reverse iontophoresis. An advantage of this may be that reverse iontophoresis has the potential of significantly enhancing the amounts of a specific substance to be extracted from the skin.

The method may be performed at a pH in a range from 5 to 10. Preferably method may be performed at a pH range from 7 to 9. According to the method of the present invention there is no restriction in which pH that may be used during sampling except for it should not be harmful in contact with the skin of an individual.

The sampling may be performed during a specified sampling time. The sampling time may be the sampling time including all the steps of the non-invasive method of the present invention. The sampling time may also refer to the step of extracting at least one endogenous substance on the area of the skin surface of the individual, or wherein the extracting step ii comprises the use of reverse iontophoresis, or the step of delivering an irritant or an allergen prior to and/or simultaneously to step ii from the matrix to estimate if said irritant or allergen trigger a response in the skin of the individual. Thus, the specific time may be a sampling time of less than 30 minutes, less than 1 hour, less than 2 hours, less than 3 hours, less than 4 hours, less than 5 hours, less than 6 hours, less than 12 hours, or less than 24 hours. The sampling time may also be during a long term, wherein a drug is delivered for treating a disease. The specified sampling time could depend on application and tentatively cover minute(s) and up to at least 48 hours or even longer.

Short application probably only reflect surface sampling, while longer application may allow for endogenous substances to be extracted from viable tissue to the surface. Sampling time do of course affect the concentrations of absorbed solutes, whereas there ratios might still be constant after sampling a sufficient time. On the other hand, with regards to a skin condition that emerges from provocation or that should be counteracted by simultaneous treatment, then the sampling time can definitely affect the outcome.

The at least one endogenous substance may be a low molecular weight substance with a molecular weight of typically up to 2000 Da, typically up to 1000 Da, typically up to 500 Da. In one embodiment the at least one endogenous substance has a molecular weight of at most 1000 Da. In another embodiment the at least one endogenous substance has a molecular weight of at most 500 Da. There is no upper limit in molecular weight of the substances that the matrix may extract, however most endogenous substances that may diffuse through skin is below 500 Da.

The at least one endogenous substance may range from hydrophilic compounds through amphiphilic compounds to hydrophobic compounds. The at least one endogenous substance may be selected from hydrophilic compounds and hydrophobic compounds. The at least one endogenous substance may be extracted with other endogenous substances or its metabolites or a mixture thereof. The at least one endogenous substance may be selected from amino acids and metabolites of amino acids or mixtures thereof. The amino acids or metabolites thereof may be selected from the group consisting of: tyrosine (Tyr), phenylalanine (Phe), trypthophan (Trp) and kynurenic acid (Kyn).

The determining step iii may be performed by quantifying the ratio between an endogenous substance that is associated with an inflammatory disease and a reference endogenous substance. For the success quantifying, the at least one endogenous substance may be at least two endogenous substances that may be compared in accordance with their abundance in the skin of a healthy individual vs an individual carrying a disease associated with e.g. inflammation.

The at least one endogenous substance may be associated with inflammatory diseases selected from the group consisting of: seborrheic dermatitis, rosacea, lupus, psoriasis, eczema, ichtyosis, skin cancer, diabetes and inflamatory bowel disease. Skin cancer may be selected from the group consisting of basal cell carcinoma, squamos cell carcinoma and melanoma. Endogenous substances, such as various endogenous amino acids, lipids, etc, and their related substances, associated with said inflammatory diseases are usually prone to be emitted through the skin of an individual.

According to a fourth aspect there is provided a use of a matrix according to the first aspect of the invention or a patch according to the second aspect of the invention for non-invasive sampling of at least one endogenous substance by extracting said at least one endogenous substance on an area of the skin surface of an individual.

The use may further comprise determining said at least one endogenous substance.

In one embodiment, there is a use according to the fourth aspect, wherein the non-invasive sampling may further comprise delivering a drug or a bioactive agent from the matrix according to the first aspect of the invention or the patch according to the second aspect of the invention to the area of the skin surface prior to and/or simultaneously extracting said at least one endogenous substance on the area of the skin surface of the individual, to trigger a response that reflects the presence of the at least one extracted endogenous substance. Thus, from the presence of the at least one extracted endogenous substance it may be estimated if the individual is affected by an inflammatory disease, e.g. cancer, inflammation, allergy, diabetes or psoriasis, preferably said at least one endogenous substance is associated with inflammatory diseases selected from the group consisting of: seborrheic dermatitis, rosacea, lupus, psoriasis, eczema, ichtyosis, skin cancer, diabetes and inflamatory bowel disease.

In another embodiment, there is a use according to the fourth aspect, wherein the non-invasive sampling may further comprise delivering a drug or a bioactive agent from the matrix according to the first aspect of the invention or the patch according to the second aspect of the invention to the area of the skin surface prior to and/or simultaneously extracting said at least one endogenous substance on an area of the skin surface of an individual, to estimate if said irritant or allergen trigger a response in the skin of the individual. Thus, the matrix and/or the patch of the present invention may be used to distinguish between an irritant, allergic or toxic effect of said endogenous substance on the individual. One of the advantages of the present invention is that it might be difficult from visual observation only to tell if a skin reaction is caused by e.g. an allergen or an irritant. Hence, the cause of the reaction will of course have implications on further actions and thus it would be helpful to have a biomarker response that identifies the one or the other. The use according to the fourth aspect of the present invention, wherein said at least one endogenous substance may be associated with inflammatory diseases that comprises inflammatory skin diseases such as psoriasis, atopic dermatitis, seborrheic dermatitis, eczema, alopecia areata, ichthyosis vulgaris or contact hypersensitivity.

The use according to the fourth aspect of the present invention, wherein said at least one endogenous substance may be associated with inflammatory diseases selected from the group consisting of: cancer, inflammation, allergy, diabetes and psoriasis.

The matrix according to the present inventive concept may be used in the therapy. For example the matrix according to the present inventive concept may be used in the treatment of inflammatory diseases, such as seborrheic dermatitis, rosacea, lupus, psoriasis, eczema, ichtyosis, skin cancer, diabetes and inflamatory bowel disease.

The matrix according to the present inventive concept may be used in diagnosis. For example the matrix according to the present inventive concept may be used in the diagnosis of inflammatory diseases, such as seborrheic dermatitis, rosacea, lupus, psoriasis, eczema, ichtyosis, skin cancer, diabetes and inflamatory bowel disease. The advantage of the present invention is that the diagnosis is non-invasive and predominantly relies on that the matrix of the present invention may extract endogenous substances which are present on the skin surface of an individual. The matrix may extract endogenous biomarkers with various properties simultaneously, i.e. the matrix may absorb both hydrophilic and lipophilic substances at the same time if present.

Further features of, and advantages with, the present invention will become apparent when studying the appended claims and the following description. The skilled person will realise that different features of the present invention may be combined to create variants other than those described in the following, however the present invention is defined by the appended claims. Features of one aspect may be relevant to anyone of the other aspects.

### SHORT DESCRIPTION OF THE DRAWINGS

FIG. 1a illustrates sequential relationship between alternative micellar and liquid crystalline phases.

FIG. 1b bicontinuous cubic phases are believed to be well described by infinite periodic minimal surfaces (IPMS) of cubic symmetry.

FIG. 2a illustrates the partial phase diagram of nonionic GMO-cationic DOTAP-H₂O (w/w).

FIG. 2b illustrates the partial phase diagram of nonionic GMO-anionic DSPG-H₂O (w/w).

FIG. 2c illustrates the partial phase diagram of nonionic GMO-cationic DOTAP-150 mM NaCl (aq) (w/w).

FIG. 3 illustrates determination of the hydration level of the skin.

FIG. 4 illustrates that higher water activity has a significant impact on skin permeability for both moderately hydrophilic and more lipophilic substances.

FIG. illustrates lipid water sorption and water activity of cubic phases FIG. 6 illustrates interfacial area per unit cell (triangles) and per unit volume (circles), respectively.

FIG. 7 illustrates K(_{bl/w}) in GMO CP-phase—concentration independence and effect of time.

FIG. 8 illustrates K(_{Q/w}) vs. interfacial area per unit volume for (top-down) K(_{bl/w}) 10; 3; 1.5; 0, 1 and 0,01.

FIG. 9 illustrates in vivo that biological variations in skin permeability may be ignored if ratios instead of actual quantities are considered.

FIG. 10 illustrates the potency of electrostatics.

FIG. 11 illustrates the effect of patch on skin hydration in vivo.

FIG. 12 illustrates how patches were applied on the forearms of test persons.

FIG. 13 illustrates effect of reverse iontophoresis vs passive extraction at different pH values 4.0, 7.4 and 9.0.

FIG. 14 illustrates flux of endogenous Trp extracted by reverse iontophoresis into electrode receiver solutions at pH 7.4.

FIG. 15 illustrates Trp/Kyn ratios at the cathode determined from the cumulative amounts (black) and corresponding fluxes (grey) at pH 7.4.

FIG. 16 illustrates the total cumulative amount of Trp and Kyn collected after 24 hours of extraction.

FIG. 17 illustrates a sampling procedure according to the present invention.

### DETAILED DESCRIPTION OF THE INVENTION

The present inventive concept will now be described more fully hereinafter with reference to the accompanying drawing, in which preferred variants of the inventive concept are shown. This inventive concept may, however, be embodied in many different forms and should not be construed as limited to the variants set forth herein, rather, these variants are provided for thoroughness and completeness, and fully convey the scope of the invention to the skilled person. Although individual features may be included in different variants, these may possibly be combined in other ways, and the inclusion in different variants does not imply that a combination of features is not feasible. In addition, singular references do not exclude a plurality. In the context of the present invention, the terms “a”, “an” does not preclude a plurality.

The present invention discloses a novel matrix and a method for non-invasive sampling of at least one endogenous substance on a skin surface of an individual. The matrix comprises at least one amphiphile, wherein the amphiphile, alone or in combination with at least one structurally related amphiphile, forms a non-lamellar liquid crystalline phase together with an aqueous polar solvent mixture, wherein it comprises a water activity of at least 0.85 in a temperature range of 20-40° C.

Especially, the present invention discloses the versatility of bicontinuous cubic liquid crystals as matrices for non-invasive topical sampling of low molecular weight endogenous substances with the GMO-water system as one embodiment.

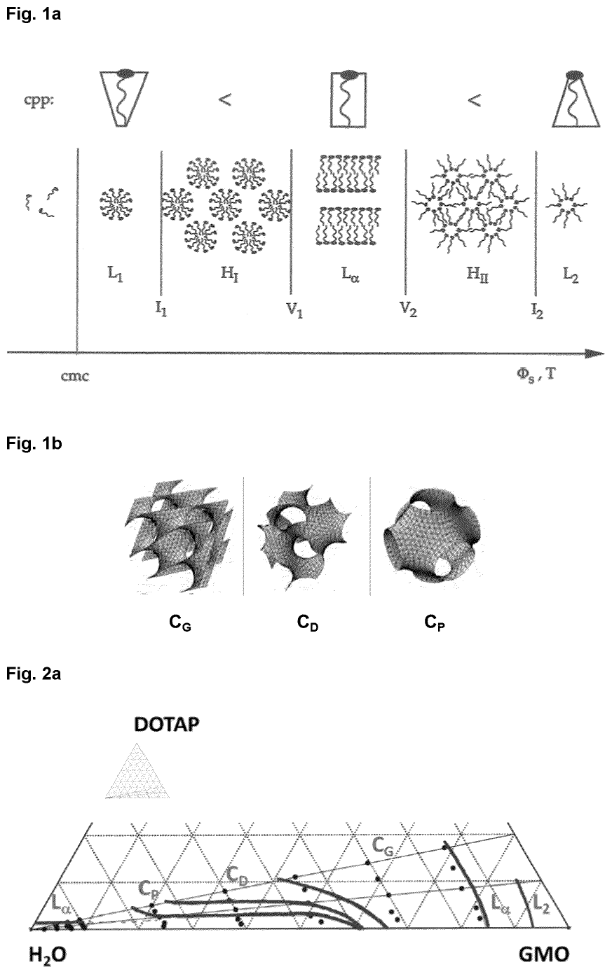

Amphiphilic lipids in water forms lyotropic liquid crystalline (LLC) phases or liquid crystalline nanoparticles (LCNPs). These are nanostructures that exhibit the typical long-range order of solids while still maintaining a certain fluidity characteristic of liquids. The LLC phases of the present invention are the non-lamellar, i.e. the hexagonal phases and the bicontinuous cubic phases. Compared to lamellar phases that may be constituted by mono-dimensional stacked bilayers, the non-lamellar liquid crystalline phases of the present invention may be selected from a cubic phase and a hexagonal phase or a mixture thereof, wherein in hexagonal phases water cylinders are surrounded by a lipid monolayer and organized in a two-dimensional hexagonal array, while in bicontinuous cubic phases two continuous but not interconnected water channels are formed by a three-dimensional and non-intersecting bilayer that extends in space superimposed over an infinite periodic minimal surface (IPMS), being the primitive body-centered lattice (Im3m), the gyroid body-centered lattice (Ia3d), and the double diamond primitive lattice (Pn3m), which are the most important IPMSs in the lipid-based systems of the present invention. FIG. 1a illustrates the sequential relationship between alternative micellar and liquid crystalline phases and their dependence on the critical packing parameter (cpp=v/al) of the amphiphile, wherein L₁ is micellar, I₁ is normal cubic, H_{I} is normal hexagonal, V₁ is normal bicontinuous cubic, L_{α} is lamellar, V₂ is reversed bicontinuous cubic, H_{II} is reversed hexagonal, I₂ is reversed discrete cubic and L₂ is reversed micellar. Further, FIG. 1b illustrates the three most common IPMS that may be seen to occur with amphiphile-water systems and they are the gyroid (CG), the double diamond (CD) and the Schwartz's primitive (CP) surfaces.

The lipid-based lyotropic liquid crystals (LLC) or liquid crystalline nanoparticles (LCNPs), are highly ordered, thermodynamically stable, internal nanostructure. Apart from the evident morphological and topological differences and the requisite of a stabilizing agent for their LCNPs formulation, non-lamellar LLC phases differentiate from the lamellar phase, e.g. hexagonal or bicontinuous cubic phases, because of the highly convoluted volumes of the lipid chains in hexagonal or bicontinuous cubic morphologies with respect to lamellar sheets, the cubosomes and hexosomes possess a larger hydrophobic volume then their liposome counterparts. For example, under the constraints imposed by the use of an identical molecular building block and NP volume, it was calculated that the hydrophobic portion of cubosomes characterized by the Im3m symmetry of the bicontinuous nanostructure and a lattice parameter of 130 A° is more than three times larger than that of liposomes of the uni-lamellar kind, and that the surface exposed to water by cubosomes may be about 60% smaller compared to that of liposomes.

The matrix may further comprise at least one ionic lipid such as an anionic lipid, a cationic lipid and a zwitterionic lipid. The matrix may also comprise further additives. Of particular interest is how incorporation of ionic lipids and addition of further additives and polar co-solvents may affect the phase behavior and then in turn the interfacial area in the cubic unit cell and subsequent endogenous substance partitioning to the lipid bilayer constituting the hydrophobic interface between separate water channels.

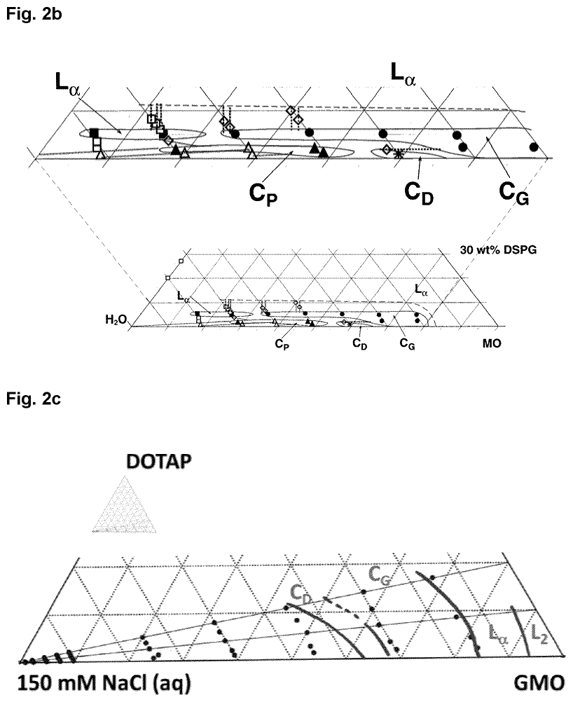

In one embodiment of the present invention the effect of incorporation of a charged lipid DOTAP on the phase behavior of GMO-water system is disclosed. Visual observations using crossed polarized light could be used to determine parts of the ternary GMO:DOTAP:H₂O phase diagram, which is shown in FIG. 2a. However, this is fine as long as you do not want to distinguish between different cubic phases. According to the present invention SAXD (=SAXS) is needed as all cubic phases are isotropic gels and look the same from visual inspection, also including the use of crossed polars.

The phase behavior of GMO in water, at 25° C., and depending on the water content it may form three different liquid crystalline phases, namely Lα, Ia3d cubic phase (CG) and Pn3m cubic phase (CD), which above 40 wt % coexists with the excess of water. DOTAP, on the other hand, forms a lamellar (Lα) phase, which swells up to 95 wt % water reaching a lattice parameter, a, of 708 Å. Further addition of water results in the formation of fully hydrated uni-lamellar vesicles coexisting with the excess of water, which has previously been observed for other charged lipids.

At low water contents (<30 wt %) the phase behaviour of GMO doped with positively charged DOTAP (up to 20 wt % of the total lipid content) was not different from what has been observed for pure GMO. In contrast to pure GMO-water system, presence of even a small amount of DOTAP resulted in a phase transition from cubic diamond (C_{D}, Pn3m) into the primitive cubic (CP, Im3m) phase as the water content was increased to 45 wt %. Coexistence between a Pn3m and Im3m phase was observed up to 75 wt % H₂O, after which only the Im3m phase was present. Increase in the DOTAP content resulted in a substantial swelling of Im3m phase, which above 95 wt % of water had a phase transition into a lamellar phase. Presence of vesicles at high water content was confirmed by PLOM measurements.

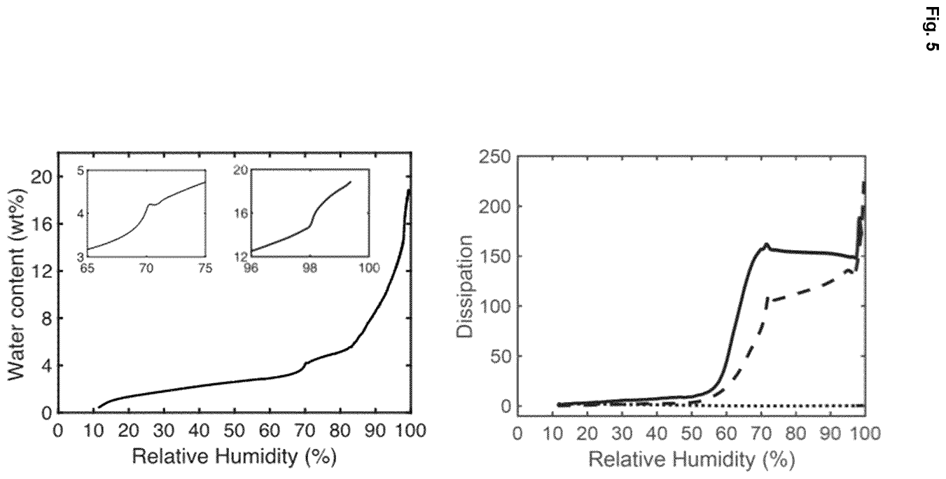

A sampling matrix of the present inventive concept may preferably be of a single phase, have a high water activity/high water content and have relatively strong mechanical properties. The higher water activity is crucial for good skin permeability as this may differ by an order of magnitude between dry and fully hydrated skin, which is illustrated in FIG. 4. Further, FIG. 4 illustrates that higher water activity has a significant impact on skin permeability for both moderately hydrophilic and more lipophilic substances. Steady state flux over excised porcine skin in vitro of metronidazole (circles, log Po/w=0.0 and methyl salicylate (squares, log Po/w=2.5) with respect to water activity of the donor vehicle. The term “donor vehicle” means the matrix in which a drug is dissolved to be delivered to the body. Thus, for extraction the “donor vehicle” would instead be a “receiver vehicle”. Based on the obtained phase diagram a sample with GMO:DOTAP content 90:10% w/w in 60 wt % water was chosen as a potential candidate for further investigation as a sampling matrix.

A humidity scan QCM-D measurements were performed on pure GMO and DOTAP as well as on GMO:DOTAP at 90:10% w/w as shown in FIG. 5 Further, FIG. 5 illustrates lipid water sorption and water activity (i.e. equilibrated at specific and continuously increasing % RH) of alternate phases obtained with (HS) QCM-D. GMO—solid line, and GMO/DOTAP 90/10 (w/w)—dotted line. The water uptake by GMO alone reveals the expected phase transitions from L2 to L_{α} around aw 0.60-0.70 (i.e. 3-4 wt % water), and then from L_{α} to bicontinuous cubic phases (first CG) at aw 0.96-0.98 (i.e. 15-16 wt % water). The water sorption of GMO/DOTAP 90/10 (w/w) is evidently very similar to that of pure GMO with respect to aw's when phase transitions occur. Several phase transitions could be observed. FIG. 6 illustrates interfacial area per unit cell (10\hat{ }3 Å², triangles) and per unit volume (m²/cm³, circles), respectively. Data collected from hydration of GMO and GMO/DOTAP 90/10 (w/w). The water activity measurements were performed on several samples in order to characterize the transitions observed with QCM-D. Thus, pure GMO with 15 wt % water, which was in a lamellar phase (FIG. 2a) had a water activity of 0.900. This corresponds to a phase obtained from GMO equilibrated in ambient air between approximately 85% RH and 95% RH. The change in the sorption curve at higher RH % was indicating transition from lamellar phase into the bicontinuous cubic gyroid (C_{G}, Ia3d) phase. A phase transition to the diamond cubic (C_{D}, Pn3m) phase could not be resolved as it appears at RH close to 100%. In case of GMO:DOTAP 90:10% w/w, water activity was measured for the samples with 30% water, which was shown to be in an Ia3d phase (see FIG. 2a), 45 wt % water (coexistence between Pn3m and Im3m phase) and 60 wt % water at which the system was in a single Im3m phase. The measured water activities were 0.962, 0.991 and 0.999 respectively. The obtained results implies that GMO and GMO:DOTAP undergoes phase transition from L₂ to a lamellar phase in a RH range 60 to 75%, transition from lamellar phase to the cubic gyroid phase in RH range between 95 and 99% followed by formation of Im3m phase.

Small angle X-ray diffraction (SAXD) measurements on GMO and GMO:DOTAP (90:10% w/w) were performed in a water content range between 15 and 99 wt %. The resulting diffractograms are exemplified in FIG. 4 Left. A clear difference may be seen between the two sample systems. While the swelling of pure GMO is limited to approximately 40 wt % water resulting in cubic diamond phase (Pn3m) with a lattice parameter, a, of around 95 Å, incorporation of 10 wt % DOTAP leads to extensive swelling of the system. Swelling curves were calculated according to a known procedure (Engblom J and Hyde ST; On the Swelling of Bicontinuous Lyotropic Mesophases; J. Phys II (France), 5 (1995) 171-190) and the change in the lattice parameter normalized by the monolayer thickness as a function of lipid volume fraction is shown in FIG. 4 Right. The results showing lattice parameters calculated from X-ray data and corresponding dimensions of the respective samples and their space group symmetry are summarized in Table 1.

TABLE 1
Data shows lipid-water composition, lattice parameter (a),
normalized lattice parameter over lipid monolayer thickness (a/l),
radius of the water channel (r), average radii of curvature (<R>),
area of the unit cell (A_{UC}), length of the water
channels (L_{w}) volume fractions of lipid (Φ_{lipid}), and the
space group symmetry of the phase for pure GMO (35 and 45 wt % H₂O)
and GMO:DOTAP (90:10% w/w) at various lipid-water contents.

| Lipid | a | | r | <R> | A_{UC} | L_{w} | Φ | |
|---|---|---|---|---|---|---|---|---|
| (wt %) | (Å) | a/l | (Å) | (Å) | (Å²) | (Å) | lipid | Sym. |
| 70 | 126.9 | 7.46 | 14.5 | 31.5 | 49671 | 251 | 0.73 | Ia3d |
| 64 | 93.5 | 5.50 | 19.5 | 36.5 | 16766 | 73.0 | 0.65 | Pn3m |
| 84.3 | 99.9 | 5.88 | 7.78 | 24.8 | 30872 | 198 | 0.88 | Ia3d |
| 70.0 | 134.1 | 7.89 | 16.2 | 33.2 | 55555 | 266 | 0.70 | Ia3d |
| 54.2 | 111.4 | 6.55 | 26.5 | 43.5 | 23803 | 87.0 | 0.56 | Pn3m |
| 54.2 | 141.3 | 8.31 | 26.2 | 43.2 | 46843 | 173 | 0.56 | Im3m |
| 40.0 | 186.6 | 11.0 | 40.1 | 57.0 | 81650 | 228 | 0.43 | Im3m |
| 25.1 | 289.2 | 17.0 | 71.5 | 88.4 | 196196 | 353 | 0.28 | Im3m |
| 10.0 | 301.2 | 17.7 | 75.2 | 92.0 | 212759 | 368 | 0.27 | Im3m |
| 10.0 | 406.4 | 23.9 | | | | | | Lα |
| 7.0 | 402.8 | 23.7 | 106.3 | 123.0 | 380471 | 492 | 0.21 | Im3m |
| 7.0 | 534.7 | 31.5 | | | | | | Lα |
| 5.0 | 708.8 | 41.7 | | | | | | Lα |

It is known that hydration increases the permeability of the skin barrier.

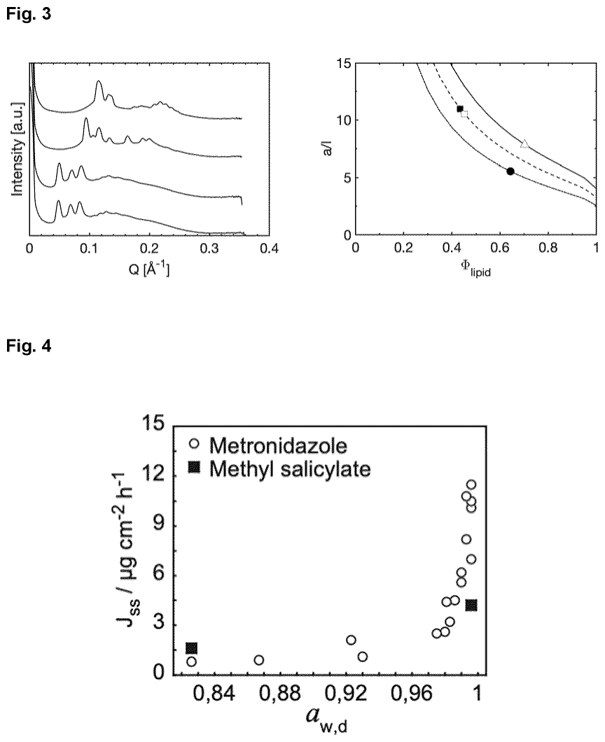

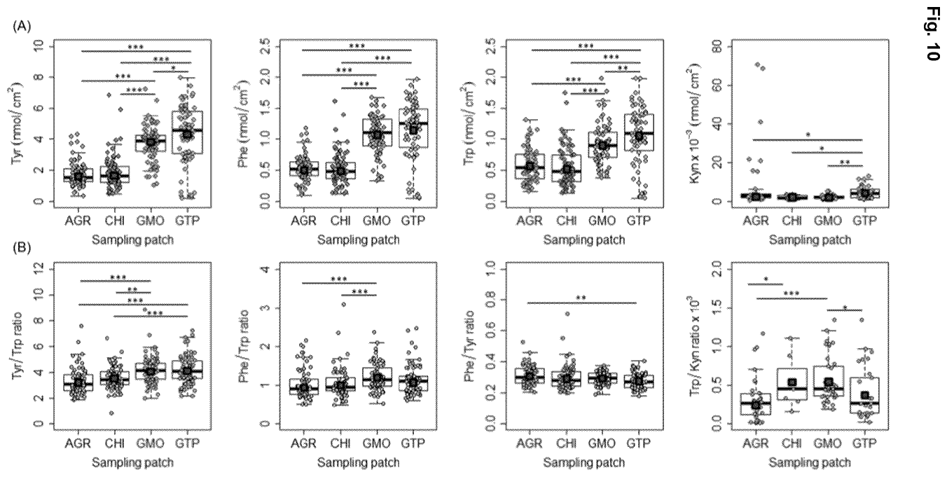

FIG. 3 illustrates determination of the hydration level of the skin. In the FIG. 3 Left, the lattice parameter (a) is determined by SAXD for two matrices of bicontinous cubic morphology (GMO-DOTAP-water (CP), and GMO-water (C_{D}), respectively) before (diffractogram 1 (Im3m) and 3 (Pn3m) from below) and after application to skin in vivo for two hours (diffractogram 2 (Im3m) and 4 (Ia3d)). Further, in the FIG. 3 Right, the water uptake by the skin is related to the change in lattice parameters of the matrices, the applied volume and the skin contact area of the matrices, and may be calculated using the models disclosed by Engblom J and Hyde ST; On the Swelling of Bicontinuous Lyotropic Mesophases; J. Phys II (France), 5 (1995) 171-190. Filled symbols—before, open symbols—after application; Theoretical swelling for spacegroup symmetry (from below): Pn3m, Im3m and Ia3d. Therefore, it is important for the sampling matrix to contain sufficient amount of water, which can hydrate the skin barrier represented mainly by the stratum corneum, SC. FIG. 9 illustrates in vivo that biological variations in skin permeability may be ignored if ratios instead of actual quantities are considered. In order to investigate the effect of topical application, a two-hour in vivo experiment was performed using a fully swollen GMO (Pn3m phase), which contains approximately 38 wt % water and GMO:DOTAP (90:10 wt %) containing 60 wt % water (Im3m phase). The SAXD results present in FIG. 3 showed that a two-hour contact with the skin resulted in a minor decrease of the lattice parameter in case of GMO:DOTAP sample (lattice parameter decreased by 8 Å). A more pronounced change was observed in GMO sample, where a two-hour skin application caused a phase transition into Ia3d. This was most likely caused by the absorption of water by the skin from the GMO cubic phase. By calculating the water loss based on SAXD data and it clearly shows that the volume fraction of water changes from 0.36 (Pn3m/before) to 0.30 (Ia3d/after), and from 0.57 (Im3m/before) to 0.55 (Im3m/after). Thus, the matrix according to the inventive concept hydrate skin to some extent with the effect of promoting extraction of endogenous substances.

FIG. 10 illustrates in vivo the superiority of lipid-based patches of the current invention (i.e., GMO and GTP) over agarose (AGR) and chitosan (CHI) hydrogels in extracting larger amounts of endogenous amino acids through skin. The data further shows that electrostatics also facilitates extraction, c.f. GMO (i.e. nonionic GMO/water CD-phase) vs GTP (i.e., anionic GMO/DOTAP/water CP-phase). potency of electrostatics.

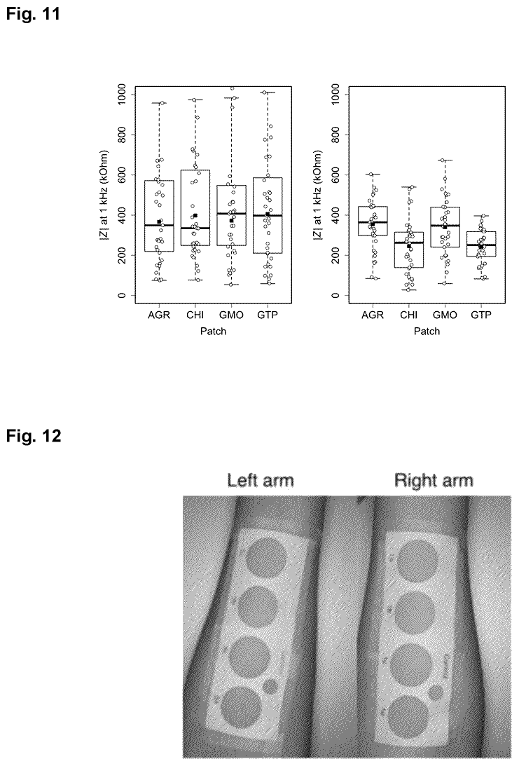

Further, FIG. 11 illustrates the effect of the four patches from FIG. 10 on skin hydration in vivo. Left: before application and Right: after 2 hours application. It can be concluded from skin impedance data that the two charged matrices, CHI and GTP, are better at hydrating skin.

The effect of electrolytes, such as sodium chloride (NaCl) on the phase behaviour of GMO:DOTAP system is also disclosed. In these experiments, 150 mM NaCl solution was added instead of Milli-Q to the dry lipid mixtures and the resulting phase diagram is shown in FIG. 2c. Thus, the addition of 150 mM NaCl resulted in strongly decreased swelling due to almost complete screening of charges. No Im3m phase could be observe in any of the samples. The excess of water was present in all above 45 wt % of NaCl (aq).

The present invention also discloses a patch that may be used as a diagnostic tool
(i) to conduct topical sampling of LMW endogenous substances which reflect biochemical reactions in the viable epidermis and dermis;
(ii) to detect the endogenous substances directly on patch or assay them off-site;
(iii) to account for differences in endogenous substance penetration rates through stratum corneum (SC) in developing diagnostic protocols based on analysis of endogenous substance ratios; and
(iv) to verify that changes of LMW endogenous substances and their ratios correlate with the levels of HMW endogenous substances in cellular models of skin disorders. For example, FIG. 12 illustrates how patches may be applied on the forearms of test persons for the tests disclosed FIGS. 9-11 according to the present application. A patch may be applied as a single patch, such as a plaster, or in an array of patches for e.g. testing the effect of different humectants, drugs, bioactive agents, irritants or allergens to the skin of an individual.

The present invention also discloses a non-invasive method for sampling of at least one endogenous substance on the skin surface of an individual (see FIG. 17), the method comprising: step i. placing a matrix or a patch according to the invention against an area of the skin surface, e.g. a forearm, of an individual (FIG. 17 B), wherein a tape strip may cover the forearm and the matrix may be placed approximately at a distance in the range of 0-30 cm from a palm of the individual on the skin of the forearm, preferably 5-20 cm from the palm, preferably 7-15 cm from the palm. The patch may have a surface area in the range of 0.5-5 cm², preferably 1-3 cm². The patch may have a thickness of 0.1-2 mm, preferably 0.5-1 mm. The patch may be of any shape, it may be circular, oval, square or rectangular, but it is not restricted to any of these shapes.

The method further comprising: step ii. extracting at least one endogenous substance on the area of the skin surface of the individual (FIG. 17 D), optionally the extracting step ii comprises the use of reverse iontophoresis; and iii. determining the presence of said at least one endogenous substance (FIG. 17 E). FIG. 17 illustrates a sampling procedure according to the present invention, wherein the extraction of endogenous substances at rest (FIG. 17 B) may be compared with extraction of endogenous substances while sweating (FIG. 17 C) and/or blood sampling for systemic levels of analytes (FIG. 17 A).
The method further comprises a step of delivering a drug or a bioactive agent prior to and/or simultaneously to step ii from the matrix, to trigger a response that reflects the presence of the at least one extracted endogenous substance. Further, the at least one endogenous substance may be extracted with other endogenous substances or metabolites thereof and the determining step iii comprises estimating a ratio between two of the extracted endogenous substances or metabolites by this the response and progression of e.g healing may be monitored in parallel to, or following the treatment (see FIG. 17 E) Thus, it may be used to estimate if the individual is affected by an inflammatory disease. Table 2 provides the compositions of the patches for the tests disclosed in FIGS. 9-11.

TABLE 2

| | Extraction formulation | | | |
|---|---|---|---|---|
| Ingredients | AGR | CHI | GMO | GMO:DOTAP |
| Water | 96 | 98 | 38 | 60 |
| Agarose | 2 | — | — | — |
| Chitosan | — | 2 | — | — |
| GMO | — | — | 62 | 34 |
| DOTAP | — | — | — | 4 |
| Water activity, a_{w} | 0.997 | 0.993 | 0.999 | 0.999 |
| Appearance | Stiff, | Sticky, | Viscous, | Slightly |
| | clear gel | viscous, | clear gel | watery, semi- |
| | | clear gel | | clear gel |

The extracted endogenous substances may be associated with inflammatory diseases, such as cancer, inflammation, allergy, diabetes and psoriasis. The non-melanoma skin cancers (NMSCs), such as basal-cell carcinoma (BCC) and squamous-cell carcinoma (SCC), are the most common and continuously growing forms of cancer. The melanoma-related skin cancers, which are less common, but more dangerous than NMSCs due to its ability to spread to other organs, have become one of the fastest-growing forms of the disease. The most crucial factor for continuous rising incidence rate is due to the increased exposure to the UV radiation. Detection of cancer at its early stage is highly important as it greatly increases the chances for patient survival. Currently, the golden standard for skin cancer diagnosis relies primarily on visual inspection of lesion followed by the tissue biopsy and staining. The correctness of skin cancer detection by visual inspection is strongly dependent on factors such as clinicians' experience and characteristics of lesions, and in fact, the accuracy of skin cancer diagnostics by visual inspection varies between 49% and 81%. Therefore, a highly common and harmless lesion type, benign nevus, may be mistaken for cutaneous melanoma, resulting in a significant amount of unnecessarily excised benign lesions. Tissue biopsy, while being the most accurate method for cancer diagnostics, suffers from several drawbacks such as invasiveness, which increases the risks of infections, high costs and long waiting times for patients. Therefore, development of alternative or complementary non-invasive methods for early-stage skin cancer diagnostics is highly desirable. Such methods would help to justify the need for surgical interventions and at the same time decrease the number of the unnecessary biopsies, which is favourable from both economical and patient perspectives. Numerous different techniques based on visual, e.g. dermoscopy, confocal microscopy, Raman spectroscopy etc., and nonvisual, e.g. electrical impedance spectroscopy, tissue dielectric constant, genomic detection of melanoma by stratum corneum stripping, evaluation of suspected lesions has been developed as supporting diagnostic methods for physicians. However, even though these systems have many advantages, there is still a number of disadvantages such as high operational costs, requirement of high expertise to analyze the data due to its complexity, difficulty to detect precancerous state, limitations in sensitivity.

Cancer affects not only the physical properties of the skin tissue, but also modify the skin chemistry, which is represented by a tremendous number of different endogenous substances including lipids, proteins, inflammatory mediators, nucleic acids and single amino acids and their metabolites. In some cases, sustained inflammation acts as the precursor for cancer, e.g. actinic keratoses and Bowen's disease are precursors for SCC3 (Sister-chromatid cohesion protein 3). Thus, the detection of inflammation endogenous substances can serve as an early warning for disease onset. Therefore, the non-invasive sampling according to the present invention of inflammation and cancer-related endogenous substances in suspected cancer lesions is an attractive approach for diagnostics of the disease.

Due to the progress in cancer research the number of endogenous substances associated with inflammation and cancer, e.g. IL-6, IFN-γ, TNF-a, enzyme indoleamine-2,3-dioxygenase (IDO), BRAF gene mutations, is continuously increasing. However, a non-invasive sampling of these high molecular weight (HMW) endogenous substances, produced in the viable epidermis, is not feasible as they cannot permeate across the skin barrier represented by the stratum corneum (SC). The barrier properties of SC are assured by its structure, consisting of corneocytes embedded in a continuous multilamellar lipid matrix, the main purpose of which is to effectively prevent the uncontrolled water loss and restrict the entrance of harmful substances. The molecular weight of inflammation endogenous substances that may be used for non-invasive topical monitoring should not be higher than 500 Da, since the permeation of compounds with higher molecular weight is strongly diminished by SC.

Since the stratum corneum is the outermost layer of the skin and acts as a front line of body defenses against environmental injuries, by arranging corneocytes in a characteristic “brick and mortar” configuration avoiding entry of exogenous materials. However, lipids may fluidize the stratum corneum facilitating the passage of drugs through the skin. In other words, the stratum corneum barrier may be affected by so-called penetration enhancers, such as fluidizing lipids, e.g. GMO, which facilitates the passage of drugs through the skin. Thus, the matrix according to the first aspect of the present invention have demonstrated useful for topical administration of drugs, and also the subsequent extraction of endogenous substances present on a skin surface of an individual.

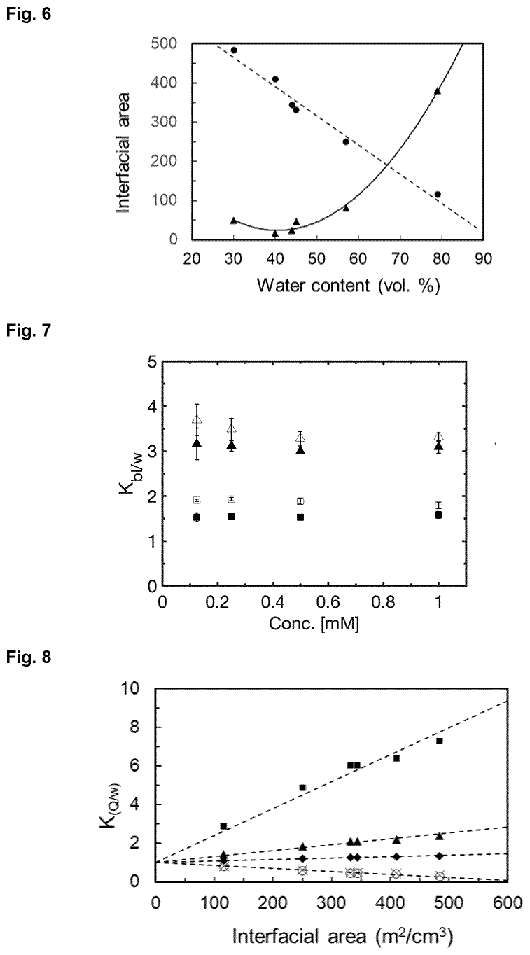

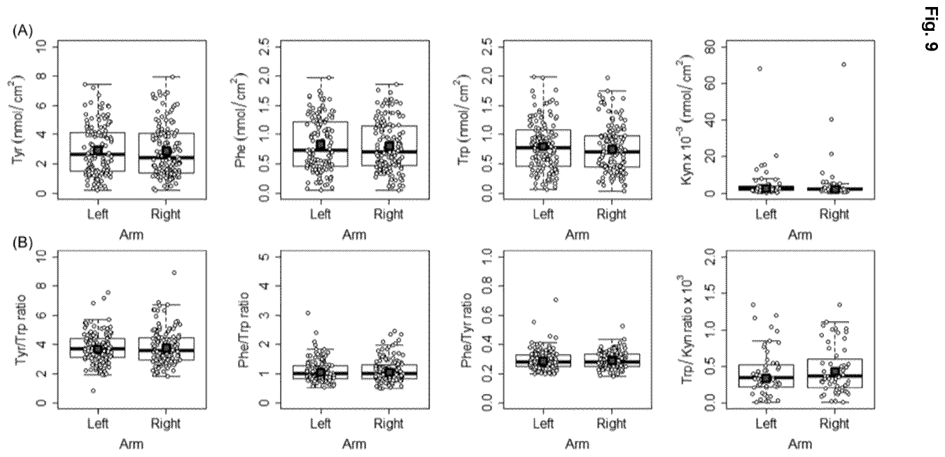

The at least one endogenous substance may be selected from hydrophilic compounds and hydrophobic compounds. The at least one endogenous substance may be extracted with other endogenous substances or its metabolites or a mixture thereof. The at least one endogenous substance may be selected from amino acids and metabolites of amino acids or mixtures thereof. The amino acids or metabolites thereof may be selected from the group consisting of: tyrosine (Tyr), phenylalanine (Phe), trypthophan (Trp) and kynurenic acid (Kyn). Partitioning of Trp and Kyn into the bilayer was investigated over two weeks by measuring the content of respective amino acid in the aqueous phase. The result is shown in FIG. 7, which illustrates a lipid bilayer-water partitioning (K_{bl/w}) of Kyn (log D_{o/w}(pH7.4)−1.9) and Trp (log D_{o/w}(pH7.4)−1.1) in the fully swollen nonionic GMO/water CD-phase, vs initial concentrations in the aqueous phase, after 7 and 14 days. Thus, it was seen that tryptophan had approximately twice as high partitioning into the cubic bilayer compared to kynurenine. This may be explained by the fact that tryptophan (at pH 7) has a slightly higher log D−1.1 compared to kynurenine, which has log D of −1.9. Further, FIG. 8 illustrates the scaling of matrix-water partioning (KQ/w) vs. interfacial area per unit volume for a solute with K(_{bl/w}) 10; 3; 1.5; 0, 1 and 0,01, respectively in a nonionic lipid/water system.

Hence, the addition of DOTAP to the GMO water system causes a phase transition from the Pn3m (diamond cubic phase) to the Im3m (primitive cubic phase), which swells up to 95 wt % of H₂O. At higher water contents, a phase transition into a lamellar phase was observed. The swelling was strongly restricted in the presence of 150 mM electrolyte solution, also, no phase transition from Pn3m into Im3m could be observed. A two-hour topical application of a fully swollen GMO:H₂O cubic phase resulted in a loss of water from the cubic phase and transition into the two-phase region (Ia3d and Pn3m). No phase transition could be observed in case of GMO:DOTAP:H₂O (36:4:60% w/w/w) initially present in Im3m phase, however, there was a small decrease in the lattice parameter. The partitioning of tryptophan (Trp) and kynurenine (Kyn), into the lipid bilayer of the fully swollen cubic phase shoed that a more hydrophobic Trp had twice as high partitioning into lipid bilayer compared to a more hydrophilic Kyn.

Further, a tryptophan-to-kynurenine ratio (Trp/Kyn) is one of the potential endogenous substance candidates for non-invasive skin cancer diagnostics. The catabolism of the essential amino acid tryptophan (Trp) along the kynurenine pathway (KP) plays a crucial role in regulation of the immune response. The conversion of Trp into Kyn along KP is catalysed by the enzymes indolamine 2,3-dioxygenase 1 and 2 (IDO1/IDO2) and tryptophan 2,3-dioxygenase (TDO). In healthy conditions, the conversion of Trp-to-Kyn is well regulated. However, in abnormal conditions, activation of IDO by proinflammatory cytokine IFN-γ leads to upregulated conversion of Trp-to-Kyn altering the Trp/Kyn ratio. It has previously been confirmed that the change in the Trp/Kyn ratio is associated with several diseases including cancer. The intrinsic physical-chemical properties of Trp and Kyn (see Table 3) make them ideal candidates for non-invasive topical sampling.

TABLE 3
Physicochemical properties of two cancer related endogenous substances.

| Endogenous | | Mw | |
|---|---|---|---|
| substance | Structure | (g mol⁻¹) | LogD* |
| Tryptophan (Trp) | 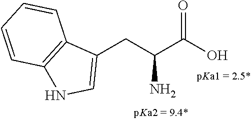 [SMILES: N[C@@H](Cc1c[nH]c2ccccc12)C(=O)O] | 204.22 | −1.1 at pH 4.0 −1.1 at pH 7.4 −1.2 at pH 9.0 |
| Kynurenine (Kyn) | 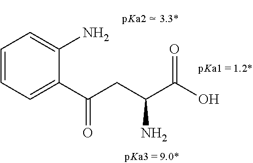 [SMILES: Nc1ccccc1C(=O)C[C@H](N)C(=O)O] | 208.21 | −2.0 at pH 4.0 −1.9 at pH 7.4 −2.2 at pH 9.0 |

*Theoretically determined values from ChemAxon software (See Supportive information)

However, the barrier properties of SC might affect their diffusion rates across the skin, resulting in a different Trp/Kyn ratio at the skin surface compared to the actual ratio at the tumour site. This together with the fact that the Trp/Kyn ratio is not the same between healthy and diseased state, represents the main challenge and makes it crucial that the extraction and quantification of these molecules from the skin proceeds in a reproducible, precise, and accurate manner. One possible solution to address this challenge is to use reverse iontophoresis, which greatly induces the transport of charged and polar substances across the skin by application of a small electric current (<0.5 mA·cm⁻²) resulting in much higher permeation rates compared to their passive permeabilities. The technique has been shown to be an effective non-invasive method for clinical and therapeutic monitoring of drugs, natural moisturizing factors (NMF) and glucose. Moreover, reverse iontophoresis was successfully applied for monitoring of prostaglandin E2 associated with cutaneous inflammation.

In another embodiment of the present invention the reverse iontophoretic extraction may be performed into receptor compartments containing either buffered or unbuffered electrolyte solution. However, from the practical point of view, it is more convenient to use an iontophoretic patch with a gel-like receptor matrix or extraction matrix, which may be easily applied onto the skin of an individual. There are several requirements, which need to be satisfied before a matrix may be considered for iontophoretic application. The matrix material should be biocompatible, non-irritant, have the ability to absorb and release molecules of interest, ability to absorb high amounts of water, tolerate mechanical stress, and have a good adhesiveness to the skin. Iontophoretic patches based on the hydrogels and synthetic polymers are among the most investigated materials due to their biocompatibility, high water content and ease of handling. However, there are several drawbacks related to hydrogels e.g., high swelling leads to a severe reduction of mechanical strength as well as low ability to incorporate hydrophobic substances. An embodiment of the present invention discloses an alternative patch to hydrogels, a patch comprising of a polar lipid glycerol monooleate (GMO) that exhibits a rich polymorphism in water. GMO is one of the most well studied polar lipids and is widely used in a number of different fields due to its nontoxicity, biocompatibility and biodegradability. At room temperature, GMO can form several different liquid crystalline phases depending on the water content. Thus, by increasing the water content from 0 to 40 wt %, GMO undergoes transition between three different liquid crystalline phases: a lamellar phase (Lα), and two bicontinuous cubic crystalline phases: Ia3d symmetry (gyroid, G) and Pn3m symmetry (diamond, D). The Pn3m cubic phase of GMO can coexist with an excess of water. One of the most attractive features of bicontinuous cubic phases is existence of highly interconnected and accessible pore network of water and lipid channels, which makes it possible to accommodate both hydrophilic and hydrophobic compounds, which is not possible with a hydrogels. Due to the existence of water channels, bicontinuous cubic phases are suitable matrices for iontophoretic applications, since ions and small hydrophilic molecules can freely move inside the water channels. Bicontinuous cubic phase of GMO has been previously employed as a vehicle for iontophoretic delivery of salbutamol. Before the present application, there are no studies on reverse iontophoresis with a bicontinuous cubic phase as a receptor matrix or an extraction matrix. Additionally, monoolein is a known permeation enhancer, which together with reverse iontophoresis may have a synergistic effect on the enhanced transport across the skin membrane, which is of great interest from the perspectives of drug delivery and extraction of endogenous substances.

Passive extraction of charged and highly polar compounds may be enhanced by means of reverse iontophoresis, which was previously shown to be an effective method for non-invasive monitoring of amino acids both in vitro and in vivo. Therefore, the extraction of Trp and Kyn was performed using reverse iontophoresis and results were compared to the corresponding passive diffusion experiments. The iontophoretic fluxes of Trp and Kyn were investigated in order to understand if they have similar permeation rates across the skin membrane. Moreover, since Trp is a part of SC NMF ‘reservoir’, it is important to consider how the extracted Trp/Kyn ratio is affected by the amount of naturally occurring Trp in the SC. The effect of pH on the reverse iontophoretic extraction was studied at three different pH, as it influences the net charge of the skin membrane affecting the magnitude of electroosmosis that is known to be a primary extraction mechanism of neutral species.

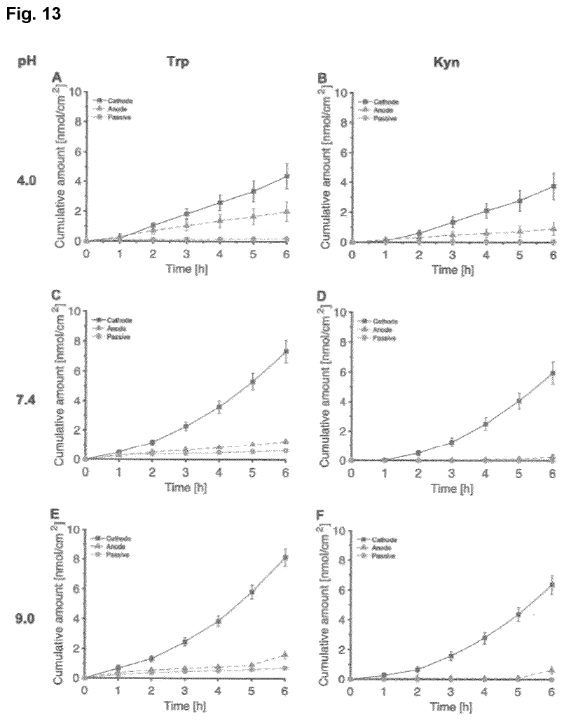

The reliability of skin cancer diagnostics based on non-invasive topical monitoring of Trp/Kyn ratio disclosed herein as a potential skin cancer endogenous substance requires the precise, well controlled, reproducible and accurate sampling of both compounds. Due to the small molecular weight, Trp and Kyn are good candidates for non-invasive monitoring as their passive permeation across the skin membrane is not restricted by the SC's ‘500 Dalton’ rule. Moreover, because of their polar nature (zwitterionic at neutral pH), these compounds are perfect candidates for extraction by reverse iontophoresis. The non-invasive reverse iontophoretic sampling of Trp and Kyn was conducted in vitro using side-by-side cells consisting of two receptor compartments (anodal & cathodal) and a subdermal compartment. Reverse iontophoretic and passive extraction experiments were performed using identical experimental conditions. In these experiments, the subdermal ‘donor’ compartment was filled with PBS buffer (pH 7.4) containing equimolar concentrations of Trp and Kyn (1 mM), while the receptor compartments were filled with a neat HEPES buffer (pH 7.4). The reverse iontophoretic extraction was performed by application of a constant current 0.3 mA (−0.4 mA/cm²) for 6 hours in total. Sampling was performed every hour by withdrawing 1 mL of receptor solution and the amount of permeated compound was determined by HPLC-UV. Iontophoretic extraction profiles at the cathode and anode were compared to those obtained from passive diffusion experiments and the results are shown in FIG. 13, which illustrates cumulative amounts of Trp and Kyn extracted passively and by reverse iontophoresis as a function of time at three different receptor solution pH values, 4.0, 7.4 and 9.0. The extraction profiles show that Trp could successfully be sampled by passive diffusion, unlike Kyn, which was detected only in samples collected by reverse iontophoresis. However, it does not mean that Kyn does not permeate across the skin barrier during passive extraction but rather that the amount of permeated Kyn was below the limit of quantification. The main reason for the higher levels of extracted Trp is due to its presence in the SC's naturally occurring reservoir of NMF. The reservoir consists primarily of amino acids and amino acid derivatives originating from proteolysis of filaggrin.

Extraction of both compounds was greatly enhanced by reverse iontophoresis compared to passive diffusion as may be seen from FIG. 13. The main extraction of Trp and Kyn occurred towards the cathode, which was expected for zwitterionic compounds as they are transported by electroosmosis acting in the anode-to-cathode direction at neutral pH. However, small quantities of both compounds were found in the anodal compartment, which was previously observed for zwitterionic compounds. Already after the first hour of current application the cumulative amount (Q) of Trp extracted towards the cathode (Q_{C}) was significantly higher compared to that of the anode (Q_{A}) or passive diffusion (Q_{C}=0.49±0.04 nmol/cm², Q_{A}=0.28±0.06 nmol/cm² and Q_{P}=0.30±0.04 nmol/cm², p=0.02 and p=0.01). The cumulative amount of Trp extracted to the anode was not significantly different compared to the passive extraction (p=0.79). The extraction of Trp towards the anode became significantly higher than passive extraction only after 5 hours of current application (Q_{A}=1.03±0.08 nmol/cm², Q_{P}=0.54±0.12 nmol/cm², p=0.007). Finally, after 6 hours, the cumulative amount of Trp extracted towards the cathode (Q_{C}=7.36±0.75 nmol/cm²) hours was almost 6 times higher compared to Trp extracted to the anode (Q_{A}=1.27±0.13 nmol/cm²), and almost 12 higher compared to the passive diffusion (Q_{P}=0.62±0.13 nmol/cm²). The extraction of Trp to the cathode was significantly higher than corresponding extraction of Kyn during first 3 hours. Once again, the higher flux of Trp is most likely due to the presence of naturally occurring Trp in the skin reservoir as a part of NMF, which causes a significant contribution to the total amount of extracted Trp. However, after 4 hours of iontophoresis, the difference between extracted Trp and Kyn became insignificant (Q_{C, Trp}=3.59±0.43 nmol/cm², Q_{C, Kyn}=2.50±0.44 nmol/cm², p=0.11), which could indicate a depletion of Trp in the skin reservoir. Further extraction by reverse iontophoresis started to sample the subdermal Trp present in equimolar concentration with Kyn. Therefore, the differences in the cumulative amounts of Trp and Kyn after 4 hours became insignificant as both compounds were extracted to the similar extent. The results are summarized in Table 4.

At neutral receptor solution pH the highest flux (J) of Trp at the anode occurred during the first hour of iontophoresis, which is most likely due to the release of endogenous Trp from the NMF skin ‘reservoir’. The following decrease in the flux could potentially be due to continuous depletion of Trp reservoir in SC. Fluxes of endogenous substances at the cathode compartment, on the other hand, showed a steady and almost linear increase suggesting that there was a continuous sampling of both Trp and, especially, Kyn (since it is not originally presented in the skin) from the subdermal compartment. The cathode fluxes of Trp and Kyn after 6 hours were much higher compared to anode and passive fluxes (p<0.001 for both Trp and Kyn). The resulting fluxes after 6 hours are summarized in Table 5.

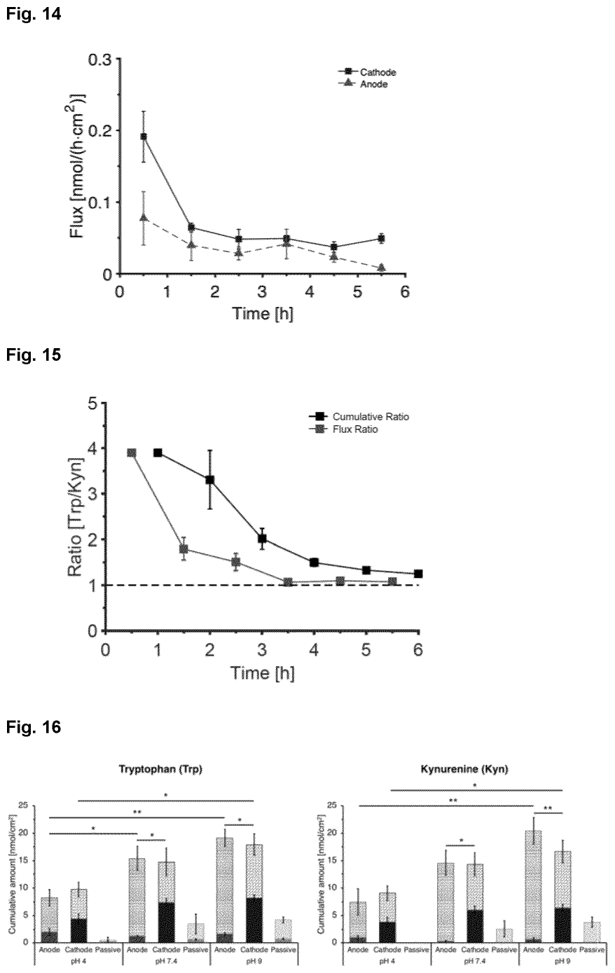

Control experiments were carried in order to ensure that the amounts of endogenously present Trp and Kyn in the skin did not have a significant contribution to the total amount of endogenous substances extracted in the previous experiments. Therefore, identical reverse iontophoretic extraction experiments were performed with an empty donor solution (i.e., neat PBS buffer pH 7.4) and the results are shown in FIG. 14. As in the previous section, Trp was extracted towards both receptor compartments, however, the main direction of extraction was towards the cathode. The presence of Trp was detected already after one hour of current application, while no Kyn was detected even after 6 hours of extraction. This is most likely due to the fact that the amount of extracted Kyn was below the limit of detection by means of HPLC-UV. Therefore, a more sensitive analysis technique is required for quantification of endogenously present Kyn.
The cumulative amount of extracted Trp to the cathode was 0.44±0.06 nmol/cm² and the anode 0.22±0.05 nmol/cm². This result implies that the contribution of endogenous Trp to the total cumulative amount of extracted Trp (at the cathode) is approximately 6%. As shown in FIG. 14, the highest flux of Trp was observed in both receptors during the first hour, after which it started to decrease indicating a depletion of the natural reservoir of Trp in the skin. In general, the obtained results suggest that compounds with a low abundance in the SC (such as Trp and Kyn) are quickly depleted from the skin reservoir by reverse iontophoresis and the following extraction is highly sensitive to the subdermal concentrations.

The direction and the magnitude of electroosmotic flow depend on the charge of the skin membrane, which may be tuned by the pH of the receptor solution. This may be used to tune the iontophoretic extraction of endogenous substances from the skin. Additionally, it is important to consider changes in the endogenous pH of the skin membrane. Thus, it is known that healthy skin has an acidic nature, which is important for its homeostasis as well as for protective purpose against microbial invasion. Nevertheless, even at healthy conditions, the pH of the skin surface can significantly vary between 4.0 and 6.5 depending on several factors such as gender, age and body site. Further, diurnal variations of 0.3 pH units of the skin surface pH have been reported. However, unlike healthy skin, chronic wounds have an alkaline pH in the range 7.5-8.9. Thus, it is reasonable to study the effect of pH on the permeability of Trp and Kyn in order to understand whether it will have an effect on the extracted Trp/Kyn ratio. Therefore, reverse iontophoretic and passive extractions of Trp and Kyn were performed at two additional pH 4.0 and 9.0. Only the receptor solution pH was varied, while the donor solution was kept at pH 7.4 to mimic the physiological conditions. The resulting cumulative amounts of Trp and Kyn are shown in FIG. 13.

The extraction of both compounds by reverse iontophoresis at pH 4 was still more efficient compared to the extraction by passive diffusion, but lower compared to reverse iontophoretic extraction at pH 7.4 (see Table 4). The cumulative amount of Trp extracted at the cathode at pH 4 was significantly lower than the corresponding amount extracted at pH 7.4 (p=0.048). However, in case of Kyn, there was no statistically significant difference in the extracted amount between pH 4 and pH 7.4 (p=0.172, Table S1). The amounts of Trp and Kyn extracted at the anode were slightly higher compared to the corresponding amounts of both endogenous substances extracted at the anode at pH 7.4. However, the difference was not significant (for Trp: p=0.660 and for Kyn: p=0.519) (see Table 4). A possible explanation to this may be due to less negative charge of the skin membrane at pH 4 (on the viable side sin is still in contact with pH 7.4), leading to a weaker electroosmotic flow. This would in turn result in a lower amount of endogenous substances extracted at the cathode, which is also observed in our results. Once again, higher amount of extracted Trp compared to Kyn was most likely caused by the release of naturally occurring Trp from the skin reservoir.

TABLE 4
The cumulative amounts of Trp and Kyn extracted by reverse iontophoresis and passive
diffusion after 6 h and Trp/Kyn ratios. The data are presented as mean ± SEM.

| | | Cumulative amount | | | ¹Ratio | | |
|---|---|---|---|---|---|---|---|
| | Endogenous | at anode | at cathode | Passive | Trp/Kyn | ²ER | |
| pH | substance | (nmol/cm²) | (nmol/cm²) | (nmol/cm²) | (after 6 h) | Anode | Cathode |
| 4.0* | Trp | 2.01 ± 0.65 | 4.39 ± 0.87 | 0.18 ± 0.18 | 1.35 ± 0.24 | 11 | 24 |
| | | (n = 12) | (n = 10) | (n = 4) | (n = 9) | | |
| | Kyn | 0.91 ± 0.44 | 3.76 ± 0.89 | <LOQ | | — | — |
| | | (n = 12) | (n = 10) | (n = 4) | | | |
| 7.4* | Trp | 1.27 ± 0.13 | 7.36 ± 0.75 | 0.62 ± 0.13 | 1.25 ± 0.03 | 2 | 12 |
| | | (n = 6) | (n = 6) | (n = 4) | (n = 6) | | |
| | Kyn | 0.25 ± 0.17 | 5.97 ± 0.73 | <LOQ | | — | — |
| | | (n = 6) | (n = 6) | (n = 4) | | | |
| 9.0* | Trp | 1.62 ± 0.26 | 8.15 ± 0.59 | 0.72 ± 0.09 | 1.29 ± 0.03 | 2 | 11 |
| | | (n = 5) | (n = 6) | (n = 4) | (n = 6) | | |
| | Kyn | 0.64 ± 0.27 | 6.38 ± 0.62 | <LOQ | | — | — |
| | | (n = 5) | (n = 6) | (n = 4) | | | |

*pH of the receptor solution, the donor solution pH was set to 7.4 in all experiments.
¹Trp/Kyn ratio was determined from the cumulative amounts of extracted endogenous substances at the cathode after 6 hours of current application (see FIG. 13).
²Enhancement ratio (ER) defined as the ratio between the cumulative amount of endogenous substance at the cathode of iontophoresis and cumulative passive amount after 6 hours (Equation 2).

Reverse iontophoretic and passive extraction profiles at pH 9 are shown in FIG. 13 and the resulting extracted amounts are summarized in Table 4. The increase in receptor solution pH to 9 resulted in the highest cumulative amounts of Trp and Kyn extracted at the cathode compared to the other two cases. The cumulative amount of Trp at the cathode was significantly higher than at pH 4 (p=0.011), but not significantly different from results obtained at pH 7.4 (p=0.814). In case of Kyn extracted at the cathode, increase in receptor solution pH from 4 to 9 did not result in a significant increase the amount of sampled endogenous substance (p=0.093). As no surprise, the difference was also not significant between pH 9 and pH 7.4. On the other hand, reverse iontophoretic extraction at the anode was not affected by the change in the receptor solution pH as no significant difference was obtained between the cumulative amounts of extracted endogenous substances.

An increase in receptor solution pH seems to enhance the passive extraction of Trp. The amount of extracted Trp after 6 hours at pH 4 was 0.18±0.18 nmol/cm² (n=4), while the corresponding cumulative amount at pH 7.4 was 0.62±0.13 nmol/cm² (n=4) and at pH 9 it was 0.72±0.09 nmol/cm² (n=4). However, the amount of extracted Trp at pH 4 was not significantly different from the cumulative amount of passively extracted Trp at pH 7.4 (p=0.095), but there was a significant difference between cumulative amounts of Trp at pH 4 and pH 9 (p=0.038). The obtained results suggest that receptor solution pH might have an effect on the passive extraction of Trp. However, in all cases except one sample collected at pH 4, the amount of passively permeated Kyn was in general below the limit of quantification. Since no other samples showed the presence of Kyn above the quantification limit, it could possibly be due to contamination.

The 6-hour flux data (determined during the last hour) for Trp and Kyn extracted by reverse iontophoresis and by passive diffusion are summarized in Table 5. In general, at pH 4 fluxes of both compounds at the cathode were lower compared to corresponding fluxes obtained at higher pH. Statistical analysis showed that receptor solution pH had a significant effect on the flux at the cathode of both compounds. Thus, in case of Trp, there was a highly significant difference in 6-hour cathode flux between pH 4 and pH 7.4 (p=0.007), as well as between pH 4 and pH 9 (p<0.001). Similar trend was observed for the flux of Kyn, the difference between pH 4 and pH 7.4 as well as between pH 4 and pH 9 was highly significant (p=0.019 and p=0.09 respectively). However, there was no significant difference in fluxes of both compounds between pH 7.4 and pH 9 (for Trp: p=0.629; for Kyn P=0.954).

TABLE 5
The extraction fluxes of Trp and Kyn after 6 hours of reverse iontophoresis
to the anode and cathode as well as the corresponding passive fluxes,
Trp/Kyn ratio calculated from the fluxes to the cathode after 6 hours
and the volume flow. The data is shown as mean ± SEM.

| | | ¹Flux | | | ²Ratio |
|---|---|---|---|---|---|
| | Endogenous | at anode | at cathode | Passive | Trp/Kyn |
| pH* | substance | nmol/(h · cm²) | nmol/(h · cm²) | nmol/(h · cm²) | (after 6 h) |
| 4.0 | Trp | 0.35 ± 0.12 | 1.03 ± 0.21 | 0.01 ± 0.01 | 1.01 ± |
| | | (n = 12) | (n = 10) | (n = 4) | 0.05 |
| | Kyn | 0.18 ± 0.09 | 0.93 ± 0.23 | — | (n = 7) |
| | | (n = 12) | (n = 10) | | |
| 7.4 | Trp | 0.24 ± 0.07 | 2.02 ± 0.19 | 0.07 ± 0.03 | 1.07 ± |
| | | (n = 6) | (n = 6) | (n = 4) | 0.02 |
| | Kyn | 0.11 ± 0.08 | 1.88 ± 0.18 | — | (n = 6) |
| | | (n = 6) | (n = 6) | | |
| 9.0 | Trp | 0.69 ± 0.23 | 2.32 ± 0.15 | 0.10 ± 0.03 | 1.17 ± |
| | | (n = 5) | (n = 6) | (n = 4) | 0.04 |
| | Kyn | 0.52 ± 0.23 | 1.99 ± 0.16 | — | (n = 6) |
| | | (n = 5) | (n = 6) | | |

*pH of the receptor solution, the donor solution pH was set to 7.4 in all experiments.
¹Flux values determined after 6 hours of iontophoresis.
²Trp/Kyn ratio was determined from the cathode fluxes of endogenous substances after 6 hours of current application.

In general, fluxes of both compounds at the anode at pH 4 were slightly, but not significantly, higher compared to the corresponding fluxes at pH 7.4 (for Kyn p=0.996, for Trp p=0.941). The anode flux at pH 4 was also slightly higher than at pH 9 during first 5 hours. However, during the last hour of extraction there was an unexpected increase in the anode flux of both compounds at pH 9. The passive flux of Kyn could not be determined as the amount of Kyn extracted by passive diffusion was below the limit of quantification. As stated previously, the increase in receptor solution pH resulted in higher amount of extracted Trp. However, the increase was not significant, for pH 4 and pH 7 (p=0.083) and for pH 4 and pH 9 (p=0.060).

In general, application of reverse iontophoresis greatly enhanced the extraction of both compounds compared to passive diffusion. This is evidenced in FIG. 14. Extraction by passive diffusion was as effective as reverse iontophoresis during the first hour due to depletion of the naturally occurring reservoir of endogenous substances in SC. The electroosmotic contribution to the total flux became progressively more important after the first two hours of current application. The pH did not show any significant influence on the iontophoretic extraction towards anode. However, the increase in receptor solution pH resulted in statistically significant increase in the extraction of endogenous substances at the cathode, which was most likely caused by the increased magnitude of electroosmotic flow.

In order for the Trp/Kyn ratio to serve as a potential endogenous substance combination for skin cancer, it is important to understand how does the extracted ratio reflects the ratio in viable tissue. For simplicity, the concentration of both compounds in the donor compartment was 1 mM. Thus, the subdermal Trp/Kyn ratio was fixed to a value of 1, in order to monitor any potential deviation from the said ratio. Beside the skin membrane properties, the extracted Trp/Kyn ratio may also be influenced by a change in pH as it has an effect on the ionisation state of both the skin and endogenous substance. This may in turn affect the permeation characteristics across the membrane and yield an altered Trp/Kyn ratio. Therefore, it is important to investigate the influence of different factors in the extracted Trp/Kyn ratio.

The amount of passively extracted Kyn was below the quantification limit at any studied pH, due to which the passive Trp/Kyn ratio could not be determined. Therefore, only the Trp/Kyn ratios were calculated from the cumulative amounts and corresponding fluxes of respective compound extracted by reverse iontophoresis at the cathode as it was the main direction of their electrotransport. The resulting ratios at pH 7.4 are shown in FIG. 15. The initial Trp/Kyn ratios at all studied pH are 2 to 4 times higher compared to the corresponding ratio in the donor compartment. This is due to the presence of endogenous Trp in the SC, which contributes the most during the first hour. Further extraction leads to a substantial decrease in Trp/Kyn ratio, which is caused by the increase in the cumulative amount of extracted Kyn across the skin membrane. Three hours of current application only partially depleted the reservoir of Trp, which has been previously reported. Therefore, longer extraction period is required in order to accurately reflect the Trp/Kyn ratio in the donor compartment. Nevertheless, the Trp/Kyn ratios determined between 4 and 6 hours of iontophoresis are very encouraging as they are in good agreement with the actual subdermal ratio. Another interesting observation is that the flux ratio was better in reflecting the subdermal ratio in all cases. This is due to the contribution from the endogenous Trp to the total cumulative amount, which makes it always higher than Kyn, resulting in Trp/Kyn>1, while the flux shows the rate of extracted endogenous substances within a specific time. With “specific time” means a specified time that is decided based on the circumstances of the sampling. Thus, the specified time may be a sampling time of 2 minutes, 15 minutes, 30 minutes, 1 hour, 2 hours, 3 hours, 4 hours, 5 hours, 6 hours, 12 hours, 24 hours or 48 hours. For example, the specified time may be a sampling time in the range of 2 minutes to 48 hours, in the range of 2 minutes to 30 minutes, in the range of 30 minutes to 1 hour, in the range of 30 minutes to 2 hours, in the range of 1 hour to 2 hours, in the range of 1 hour to 6 hours, in the range of 1 hour to 12 hours, in the range of 6 hours to 12 hours, in the range of 6 hours to 24 hours, in the range of 6 hours to 48 hours, in the range of 12 hours to 24 hours, in the range of 12 hours to 48 hours.

Zwitterions, unlike charged molecules, do not have a preferable extraction pathway, but their extraction towards the cathode is reinforced by electroosmosis. Therefore, while enhancing extraction at the cathode, electroosmosis (at pH>skin pI) retards the extraction of anions and zwitterions towards the anode as they have to move against the solvent flow. This may result in a build-up of zwitterions moving towards the anode and against the electroosmotic flow. Therefore, the skin membrane that faces the anodal compartment could potentially contain higher amounts of zwitterionic compounds compared to the skin membrane facing the cathodal compartment. In order to further investigate the effect of electroosmosis, the post iontophoretic passive extraction experiments were performed for another 18 hours.

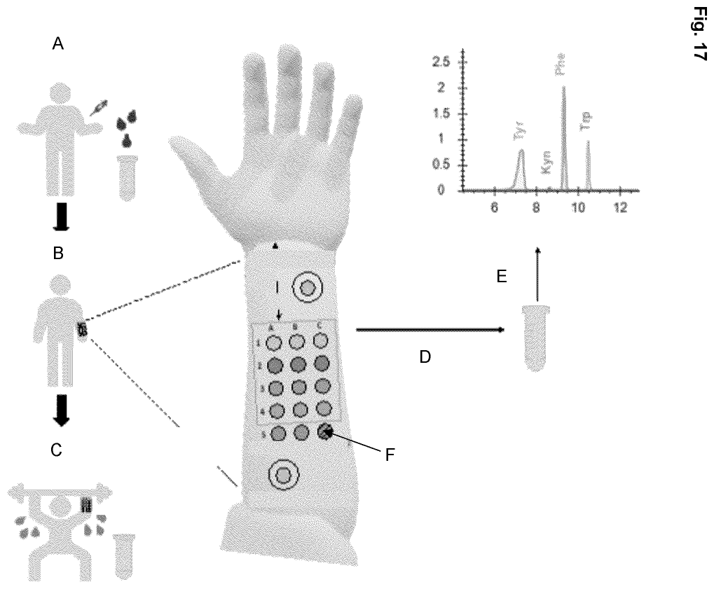

The effect of receptor solution pH on the cumulative amount of endogenous substances extracted from the skin membrane during post iontophoretic passive extraction as well as from passive control experiments is shown in FIG. 16. It may be seen that pH had an effect on the amount of extracted endogenous substances i.e., the amount of passively permeated endogenous substances increased with increase in receptor solution pH. Moreover, in post iontophoretic experiments, there was an increase in the amount of endogenous substances passively extracted into anode compartment with increased pH. In order to determine the amount of endogenous substances obtained during the post iontophoretic step, the cumulative amount of endogenous substances extracted during 6 hours of reverse iontophoresis were subtracted from the total cumulative amount. The results are summarized in Table 6.

No significant difference in the cumulative amount of Kyn extracted either at the anode or at the cathode was observed between pH 4 and pH 7.4 (for anode p=0.087, for cathode p=0.218). In contrast to Kyn, there was a significant difference in the amount of Trp extracted at the anode at pH 7.4 compared to pH 4 (p=0.012), while the difference was not significant for the extraction at the cathode (p=0.480). Increase in pH to 9 resulted in highly significant difference in the extracted amounts of both compounds into either compartment compared to pH 4 (Kyn anode p=0.005, Kyn cathode p=0.028, Trp anode=0.001 and Trp cathode p=0.047). On the other hand, there was no significant difference between pH 7.4 and pH 9. The effect of pH was also investigated by comparing the normalized quantities of endogenous substances extracted into anode and cathode at the same pH. At pH 4, there was no significant difference between the anode and cathode in the amount of extracted endogenous substance (Kyn p=0.668, Trp p=0.674). As pH increased to pH 7.4, anodal compartment contained significantly higher amount of endogenous substances compared to cathodal compartment (Kyn p=0.037, Trp p=0.022). Further increase in receptor solution pH to 9 had even more pronounced effect on the extraction at the anode (see Table 6) yielding highly significant difference in the normalized amount compared to cathode (Kyn and Trp p=0.001). Thus, the pH of the receptor solution during reverse iontophoresis has a strong impact on the post-iontophoretic passive extraction as it regulates the charge of the skin membrane, which in turn regulates the magnitude of the electroosmotic flow.

The post iontophoretic Trp/Kyn ratios (see Table 6) were lower compared to the ratios obtained after 6 hours of reverse iontophoresis. This could potentially be due to the successive depletion of endogenous Trp during the initial stage, which minimized its contribution to the post iontophoretically extracted Trp. Therefore, the ratio of Trp and Kyn in the skin membrane in post iontophoretic experiment was expected to be close to 1. Additionally, the normalized amount of passively extracted Kyn was slightly higher compared to the amount of extracted Trp at pH 7.4 and at pH 9, resulting in Trp/Kyn ration slightly below its hypothesized ratio in the skin membrane. which could be due to the fact that Kyn is more polar compared to Trp (Table 3) and therefore has a higher tendency to leave the lipophilic skin membrane than Trp.

TABLE 6
The normalized post iontophoretic cumulative amounts of Trp and Kyn as well
as Trp/Kyn ratio extracted after 24 hours. Data are shown as mean ± SEM.

| | | Normalized cumulative amount* | | | Ratio (Trp/Kyn)** | | |
|---|---|---|---|---|---|---|---|
| | Endogenous | at anode | at cathode | Passive | at | at | |
| pH | substance | (nmol/cm²) | (nmol/cm²) | (nmol/cm²) | anode | cathode | Passive |
| 4.0 | Trp | 6.22 ± 1.49 | 5.36 ± 1.32 | 0.49 ± 0.49 | 1.19 ± 0.17 | 1.05 ± 0.03 | 1.81 |
| | | (n = 11) | (n = 10) | (n = 4) | (n = 10) | (n = 9) | (n = 1) |
| | Kyn | 6.56 ± 2.40 | 5.31 ± 1.35 | 0.27 ± 0.27 | | | |
| | | (n = 11) | (n = 10) | (n = 4) | | | |
| 7.4 | Trp | 14.13 ± 2.25 | 7.38 ± 2.57 | 2.87 ± 1.66 | 0.99 ± 0.02 | 0.86 ± 0.08 | 1.27 |
| | | (n = 6) | (n = 6) | (n = 4) | (n = 6) | (n = 6) | (n = 2) |
| | Kyn | 14.35 ± 2.33 | 2.12 ± 8.37 | 2.59 ± 1.50 | | | |
| | | (n = 6) | (n = 6) | (n = 4) | | | |
| 9.0 | Trp | 17.52 ± 1.58 | 9.77 ± 1.95 | 3.50 ± 0.48 | 0.89 ± 0.02 | 0.95 ± 0.02 | 1.22 ± 0.19 |
| | | (n = 5) | (n = 6) | (n = 4) | (n = 5) | (n = 6) | (n = 4) |
| | Kyn | 19.90 ± 2.45 | 10.29 ± 2.07 | 3.81 ± 0.93 | | | |
| | | (n = 5) | (n = 6) | (n = 4) | | | |

*The amount extracted during first 6 hours were subtracted from the cumulative amount after 24 hours
**Trp/Kyn ratio determined from the normalized cumulative amounts

Another objective of the present intention was to investigate the potency of a liquid crystalline cubic phase formed by GMO and water to serve as a matrix for iontophoresis. In order to keep experimental conditions as similar as possible to the previously described experiments, the same horizontal side-by-side cells were used. A fully swollen cubic phase (Pn3m space group) was added into a custom-made sample holder ensuring the formulation was in contact with the skin. In order to prevent contact between the cubic phase and the electrodes, 2 mL of HEPES buffer (pH 7.4, 60 mM NaCl) was added to each receptor compartment. Sampling was performed by withdrawing 1 ml of receptor solution and replacing it with a 1 mL of empty buffer every hour for 6 hours. After the last sampling, the experiment was terminated, and the cubic phase was collected for further analysis. The resulting cumulative amounts obtained from the matrix are summarized in Table 6, and illustrates that both Trp and Kyn could be successfully extracted using a cubic phase as a receptor matrix. In line with previous observations, endogenous substances were primarily extracted at the cathode, even though small quantities were found in the cubic phase at the anode. After 3 hours of reverse iontophoresis detectable amounts of endogenous substances were presented in the cathode receptor solution. This means that endogenous substances were “escaping” from the cubic phase into the buffer solution. The total cumulative amounts of extracted endogenous substances are summarized in Table 7, calculated as the sum of the cumulative amount in the receptor solution after 6 hours of extraction and the amount of endogenous substances in the cubic phase. It is important to point out that partitioning of Trp and Kyn into cubic bilayer was not taken into account during the calculation of the extracted amounts of respective endogenous substance. The amounts were determined based on the volume of the receptor matrix (100 μL). The total extracted amount at the cathode was not significantly different to the amount extracted into pure buffer (for Kyn p=0.192 and for Trp p=0.538).

TABLE 7
Cumulative amount of tryptophan and kynurenine extracted by reverse iontophoresis into the cubic
phase and Trp/Kyn ratio in the cubic phase as well ratio from the total cumulative amount.

| | Cumulative amount | | | | Ratio Trp/Kyn | |
|---|---|---|---|---|---|---|
| | Cubic | Cubic | Total | Total | at | at |
| Endogenous | at anode | at cathode | at anode | at cathode | cathode | cathode |
| substance | [nmol/cm²] | [nmol/cm²] | [nmol/cm²] | [nmol/cm²] | cubic | total |
| Trp | 0.68 ± 0.05 | 5.29 ± 0.48 | 0.68 ± 0.05 | 6.57 ± 1.00 | 0.94 ± 0.04 | 0.86 ± 0.05 |
| | (n = 3) | (n = 5) | (n = 3) | (n = 5) | (n = 5) | (n = 5) |
| Kyn | 0.28 ± 0.28 | 5.62 ± 0.45 | 0.28 ± 0.28 | 7.53 ± 0.83 | | |
| | (n = 3) | (n = 5) | (n = 3) | (n = 5) | | |

The Trp/Kyn ratio obtained from the amounts of endogenous substances in the cubic phase at the cathode could correctly reflect the ratio in the subdermal compartment (see Table 7). However, the ratio obtained from the total cumulative amount of respective endogenous substance was slightly lower compared to the ratio in the donor solution. This was due to the higher amount of extracted Kyn into and out of the cubic phase over 6 hours of reverse iontophoresis compared to Trp. Additionally, since Trp is slightly more hydrophobic compared to Kyn, its partitioning into the cubic bilayer should be higher compared to Kyn, which would result in its retarded extraction from the cubic phase. Nevertheless, the obtained results with a cubic phase as a receptor matrix for iontophoretic extraction are very encouraging.

Since the stability of the cubic phase is crucial for its real application, it was import to investigate how the cubic phase would withstand the application of a current (0.4 mA/cm²) during 6 hours as well as to assure that accumulation of endogenous substances does not result in a phase change. Small angle X-ray diffraction was used to inspect any potential changes in the cubic phase. A fully swollen GMO:H₂O cubic phase (Pn3m space group) was used as a control. The effect of current on the phase stability was investigated by performing a SAXD measurement on a cubic phase collected after 6-hour iontophoretic experiment without addition of endogenous substances. In order to investigate the effect of endogenous substances on the phase stability, a pure cubic phase was doped with 1 mM Trp and 1 mM of Kyn, which is much higher than physiological concentrations, as well as the concentrations obtained after extraction. Finally, small quantities of each cubic phase (at the anode and at the cathode) were collected after extraction experiment to investigate the combination of current application and partitioning of endogenous substances on the phase stability. It is well known that at fully swollen conditions GMO forms Pn3m phase with a characteristic peak ratio: √2, √3, √4, √6, √8, √9, √10, etc., which could also be observed in this case with a resulting lattice parameter of 93.5 Å. The application of a current at its maximum allowed intensity (0.5 mA/cm²) for in vivo applications did not cause any phase shift. However, a slight shift towards higher q values could be observed, which corresponds to a small decrease in the lattice parameter from 93.5 Å, observed for control, to 91.0 Å. Addition of 1 mM of Trp and 1 mM of Kyn also did not result in any phase transition, but in contrast to the current, there was a slight shift to lower q values. This implies that incorporation of Trp and Kyn into the cubic bilayer phase resulted in increase of the lattice parameter. The application of the current together with accumulation of Trp and Kyn in the cubic phase during the reverse iontophoretic caused a further shift to lower q values resulting in further increase in the lattice parameter to 97.1 Å. These observations suggest that it is the partitioning of Trp and Kyn into bilayer and no the current that causes the swelling of the cubic phase indicated by increase in the lattice parameter. No difference could be observed between the cubic phase facing the anode compartment compared to the cathode compartment. In all cases the peak ratios were characteristic to that of Pn3m space group. All in all, the obtained results indicate that neither accumulation of endogenous substances nor the current passage had a significant effect on the stability of the cubic phase, which is highly promising for further applications.

Thus, the present invention discloses that the use of reverse iontophoresis as a non-invasive technique may enhance the extraction of Trp and Kyn compared to their passive extraction. The present disclosure shows how the Trp/Kyn ratio extracted by reverse iontophoresis may reflect the subdermal Trp/Kyn ratio. Reverse iontophoretic and passive control extraction experiments of Trp and Kyn were carried out across mammalian skin in vitro using horizontal side-by-side cells. As the pH of the extraction medium has an influence on the charge of the skin membrane, which in turn affects the magnitude of electroosmosis, extraction experiments were performed at three different receptor solution pH (4.0, 7.4 and 9.0). However, the donor solution pH was kept to 7.4 throughout all experiments, at which both compounds are present in zwitterionic form (see Table 7) and therefore are expected to be extracted mainly towards the cathode by means of electroosmosis. Additionally, reverse iontophoretic experiments were carried out with a fully swollen liquid crystalline cubic phase, i.e. GMO:H₂O˜60:40 wt %, as a receptor in order to test its potency to serve as a suitable iontophoretic matrix for sampling of endogenous substances. Small angle X-ray diffraction experiments were performed on a cubic phase before and after iontophoresis in order to investigate any potential changes in the cubic phase, which could be caused by the current application.

Bicontinuous cubic liquid crystals comprise a large interfacial area separating their interconnected polar and apolar domains which makes them susceptible to accommodate various types of solutes, being hydrophilic as well as amphiphilic or lipophilic. According to the present invention it is an object to evaluate their potential use as matrices for non-invasive topical sampling of low molecular weight endogenous substances from the skin surface. We adopt the extensively studied glycerol monooleate (GMO)-water system as the base and introduce a structurally related cationic lipid, dioleyl trimethylammonium propane (DOTAP), to moderate the cubic structure and further benefit from electrostatic attraction in extracting charged solutes. The targeted solutes are Tryptophan (Trp) and its metabolite Kynurenine (Kyn), two amino acids known to be associated with several diseases including inflammation and skin cancer.

Thus, the present invention discloses an extensive swelling of the GMO-water system and formation of a third cubic phase (Im3m) in presence of DOTAP. It is also disclosed that the cubic phases in matrix of the present invention all form at rather extreme water activities, e.g. aw>0.9. Physiological salt concentrations counteract the electrostatic effect on swelling and prevent formation of the Im3m phase, while wearing the matrix on skin in vivo for several hours only induce a marginal decrease in lattice parameters, most probably an effect of water uptake by the skin tissue. Still, presence of DOTAP also at high salt results in increased swelling of both the Ia3d and the subsequent Pn3m cubic phases.

The standard octanol/water partitioning identifies both Kyn and Trp as rather hydrophilic substances (log Do/w(pH7.4): −1.9 and −1.1/Do/w (pH7.4): 0.0 and 0.1 for Kyn and Trp, respectively), while the partitioning between a fully swollen Pn3m cubic phase (GMO/water) and water is more in favour of the cubic phase (log Kcub/w: 0.1 and 0.3/Kcub/w: 1.3 and 2.2 for Kyn and Trp, respectively). This could be attributed to the large interfacial area between the interconnected polar and apolar domains of almost 500 m²/cm³ where the bilayer/water partitioning is calculated to log Kcub/w: 0.2 and 0.5/Kcub/w: 1.5 and 3.0 for Kyn and Trp, respectively. In vivo studies have also shown that addition of DOTAP has a positive effect on the extraction capabilities for Trp and Kyn, as well as other related amino acids, despite the fact that the significant swelling of the Im3m cubic phase and the related increase in interfacial area per unit cell leads to a reduction of the interfacial area per unit volume to about 250 m²/cm³. This is a strong argument for the fact that electrostatic attraction also plays a vital role in the extraction process. Evidently, the matrix comprising bicontinuous cubic liquid crystals constitute a promising and versatile platform for non-invasive extraction of endogenous low molecular weight substances through skin, where the interfacial area per unit volume in a matrix, as well as incorporation of cationic or anionic molecules at the interface, may be used to optimize extraction of particular solutes by the matrix.

It will be appreciated that the present inventive concept is not limited to the variants shown. Several modifications and variations are thus conceivable within the scope of the invention which thus is defined by the appended claims.

### Experimental Details

### Materials

Glycerol monooleate (GMO, RYLO™ MG 19 Pharma, monoglyceride content >95% w/w, Mr=356.6 g/mol) was provided by Danisco Cultor (Brabrand, Denmark), cationic lipid 1,2-dioleoyl-3-trimethyl-ammonium-propane (DOTAP) was purchased from Avanti Polar Lipids Inc. (Alabama, US). Lipids were used without further purification. Amino acid L-tryptophan (Trp, 98% HPLC) was purchased from Sigma-Aldrich (Shanghai, China) and L-kynurenine (Kyn, metabolite of tryptophan, 98% HPLC) was purchased from Sigma-Aldrich (Buchs, Switzerland). NaCl were obtained from Sigma-Aldrich (St. Louis, MIO, USA and Fisher Scientific (Loughborough, UK), sodium phosphate dibasic (NaH₂PO₄·H₂O) was obtained from Merck (Darmstadt, Germany and Fisher Scientific (Loughborough, UK), potassium phosphate monobasic (KH₂PO₄) was purchased from Sigma-Aldrich (Tokyo, Japan), HEPES (N-2-hydroxyethyl-piperazine-N-2-ethanesulfonic acid) was purchased from Acros Organic (Geel, Belgium). Silver chloride (AgCl, metal basis >99.99% purity) and silver (Ag) wire 1 mm Ø (>99.99% purity) were purchased from Sigma-Aldrich (Gillingham, UK). Ethanol (100% v/v) and methanol of HPLC grade were purchased from VWR International (Fontenay-sous-Bois, France). LiCl (p.a. quality) was obtained from Sigma Aldrich (St. Louis, MIO, USA). Close to saturated LiCl solution was prepared by mixing the excess amounts of LiCl salt in water for several days and filtering the final saturated solution two times in order to remove the excess of LiCl salt. Glycerol monooleate (GMO) (RYLO™ MG 19 Pharma, Batch nr. 4010989490, monoglyceride content >95% w/w, Mr=356.6 g/mol) was kindly provided by Danisco Cultor (Brabrand, Denmark). PBS buffer (130.9 mM NaCl, 5.1 mM Na₂HPO₄ and 1.5 mM KH₂PO₄, pH of 7.4) and HEPES buffer (10 mM HEPES, 60 mM NaCl) were prepared in Milli-Q water (resistivity ≥18.2 MΩ·cm) and degassed by sonication for one hour prior to use. Methanol of HPLC grade was obtained from VWR international (Lutterworth, UK).

### Sample Preparation for Phase Study

GMO:H₂O samples. Solid GMO was firstly melted in a water bath tempered at 45° C., after which the appropriate amounts (0.1 g) of melted GMO were weighted in glass vials. These were then stored in a freezer (−20° C.) for 30 minutes or until GMO had crystallized. GMO samples with fixed water content in the range between 10 and 60% (w/w) were prepared at room temperature (21±0.3° C.) by addition of the required amount of water. Vials with the desired GMO:H₂O composition were sealed and centrifuged 6 times at 1000 g for 5 minutes. The resulting samples were then stored in dark place at room temperature until equilibration had occurred, usually within a week. Samples prepared by direct addition of Milli-Q water in excess equilibrated within 2 days. GMO:DOTAP:H₂O samples. Two sets of GMO:DOTAP compositions (97.5:2.5, 95:5, 90:10, 85:15 and 80:20% w/w) with 10 samples per each lipid composition were prepared by mixing appropriate amounts of GMO and DOTAP dissolved in ethanol. The desired volumes of lipid mixtures were then transferred into 1.5 mL glass vials. Solvent was evaporated using a GeneVac system at 35° C. (EZ-2 Plus Evaporating System, Genevac LTD., UK) and samples were further dried under vacuum for overnight or longer. Vials with no visible lipid residuals on the walls, were placed in the freezer (−20° C.) until crystallization. Dried lipid cakes from the first set were hydrated by addition of the required amounts of Milli-Q water in the range between 15 to 99% (w/w). The second set of samples was prepared in exactly the same way except that 150 mM NaCl solution was used instead of Milli-Q water. This was done in order to investigate the effect counterions (Cl—) on the swelling behavior of GMO:DOTAP. Final mixtures were centrifuged 6 times at 1000 g for 5 minutes and then stored in dark place at room temperature for at least 3 weeks before analysis with X-ray diffraction.

### Small Angle X-Ray Diffraction

Small angle X-ray diffraction (SAXD) was used for phase characterization. All SAXD measurements were carried out on Xeuss 3.0 SAXS/WAXS laboratory-based instrument (Xenocs, France) at Malmö University (Malmö, Sweden). In this instrument, the X-ray beam is generated by Cu K_{α} radiation source (λ=1.541 Å). All samples were measured in an ambient environment at 25° C., with a temperature-controlled Peltier gel-holder stage using an O-ring as a spacer between two Kapton films (DuPont™ Kapton®, 0.013 mm thickness, Goodfellow, England). The diffraction data was collected by Pilatus3 R 300K hybrid photon counting detector with a sample-to-detector distance (STDD) of 800 mm and 1700 mm. These STDDs covered the q-range 0.0002≤q (Å-1)≤0.36, where q is the scattering vector defined as

$$\vert q \vert = q = \frac{4 π}{λ} sin ( \frac{θ}{2} )$$

and θ is the scattering angle. One-dimensional (1D) data was obtained by azimuthal averaging of 2D-diffraction pattern and scattering intensity was corrected for background scattering and normalised to direct beam. The exposure time in most cases was 30 minutes for each sample.

### Humidity Scanning QCM-D

The hydration of the lipid films was investigated by using humidity scanning QCM-D. The technique, besides being a highly accurate for determination of a mass of materials adsorbed on piezoelectric quartz sensor based on Sauerbrey methodology, also provides information about the viscoelastic properties of the adsorbed material. It works by applying an oscillating potential on a quartz crystal and monitoring the frequency of the resulting oscillating shear motion, which generates an acoustic wave. The resonance condition occurs when the wavelength of the resulting acoustic wave is an odd integer of the quartz sensor's thickness. The information of the mass of the adsorbed material is obtained from the resonance frequency. The mass of the adsorbed material may be determined using the Sauerbrey equation, i.e. Eq. 1, under the assumptions that the mass of the material is small compared to the mass of the crystal and that the material is rigidly adsorbed and homogenously distributed over the active area of the crystal.

$$- \frac{Δ f}{n} = \frac{2 {f}_{0}^{2} {m}_{f}}{{Z}_{q}} \qquad Eq . 1$$

The Sauerbrey equation describes the relationship between the negative frequency change Δf, normalized per overtone n, and the product of the areal film m_{f}(kg·m⁻²) and the fundamental resonance frequency f₀ of the quartz sensor (˜5 MHz) normalized by the acoustic impedance of quartz Z_{q} (8.8·10⁶ kg·m⁻²·s⁻¹). In addition to the areal masses of the films obtained from the QCM-D experiments, the films are also described by their estimated thicknesses. The thickness of a dry film, d may be calculated from the areal mass of the dry film d=m_{f}/ρ, where ρ is the density of the dry film. The density of a dry lipid film constituting mostly of GMO in this work is assumed to be 0.94 g·cm⁻³. However, it is important to note that this is only an estimation of the film thickness. As mentioned earlier, the QCM-D technique also monitors the dissipation, D, which is related to the decay time of the oscillating resonator when the alternating potential is turned off. The viscoelastic properties of the film adsorbed on the quartz crystal has strong impact on its dissipation energy, which is related to the decay time. Therefore, dissipation provides information about the rheological properties of the film as well as complementary data during the hydration process.
A q-sense QCM-D E4 with humidity module QHM 401 and AT-cut SiO2 (QSX 303, 5 MHz) sensors from Biolin Scientific AB (Gothenburg, Sweden) were used in this work. The humidity module is equipped with a Gore membrane, which separates the flowing solution from the sensor, allowing only the water vapors from the solution to diffuse across the membrane and regulate the RH above the film coated on the surface. New sensors were gently washed with ethanol and Milli-Q water and dried by the flow of nitrogen, while used sensors were cleaned according to the cleaning protocol described in the q-sense guidelines manual (cleaning protocols B for QSX 303). No difference was observed in measurements performed with new and reused sensors. Lipids (GMO and DOTAP) were dissolved in ethanol in appropriate ratios so that the final concertation was 8 mM. Humidity scan QCM-D experiment was initiated by measuring the uncoated sensor in a dry N₂ atmosphere at 25° C. After that, sensors were coated with a lipid film by spin-coating where 10⁻²⁰ μL of lipid solution was applied once on the surface of the sensor. In a study made by Bjorklund et. al. it was found that film thickness is primarily dependent on the concentration and not on the number of solution applications. The coated sensors were then dried overnight in vacuum and then placed back into the humidity module. The measurements were initiated by firstly flowing dry N₂ gas until a stable baseline was observed (usually 30 minutes). After that, N₂ gas flow was stopped, and hydration experiment was performed according to a literature procedure. In brief, film hydration measurement was carried out by a continuous and controlled dilution of saturated LiCl solution that was floating through the humidity chamber. Since only the water vapors can pass across the Gore membrane, the RH above the sensor could be continuously regulated by adjusting the water activity aw of the floating LiCl solution (a_{w}=RH/100%).
Partitioning of Trp and Kyn into Lipid Bilayer

The partition experiment of Trp and Kyn into lipid bilayer of a cubic phase was done in accordance with the method described in the prior art, where the authors investigated the lipid bilayer/water partition of a model drug clomethiazole. Four concentrations of Trp and Kyn in a range 0.125 mM to 1 mM, corresponding to lipid:Trp(Kyn) ratio in the range 7200:1-900:1, were prepared in Milli-Q water in order to investigate the effect of concertation on the partitioning. The fully swollen cubic phases (˜0.25 g) were prepared by mixing the appropriate amount of GMO with an excess of Milli-Q water (1:1 weight ratio) in a 1.7 mL glass vials and left to equilibrate for 7 days. When samples were equilibrated (typically within 2 days a glass-clear and non-floating cubic phase was formed), each vial was checked between cross polarizers to ensure that no birefringence could be observed. The excess of water was then removed and 500 μL of solution with different concentrations of Trp/Kyn (molar ratio 1:1) were added to each vial. Final samples were allowed to equilibrate with respect to Trp and Kyn partitioning between the lipid bilayer and the aqueous phase. First sampling was performed after one week of equilibration by withdrawing 50 μL of the aqueous phase. Second sampling was performed in identical manner after 2 weeks of equilibration to investigate the time aspect on the partitioning. All samples collected during the partition study were diluted to a final volume 500 μL with Milli-Q water and filtered with a 0.2 μm syringe filters (13 mm PTFE membrane, VWR International, USA) prior to the HPLC-UV analysis. All partition experiments were performed in triplicates.

### Bilayer Partition Coefficient

The cubic liquid-crystalline phase consists of two domains, a lipid bilayer domain and a water domain. The lipid bilayer/water partition coefficient, K_{bl/w}, may be defined as

$${K}_{b 1 / w} = \frac{{[ X ]}_{b 1}}{{[ X ]}_{w}} \qquad Eq . 2$$

where [X] is the concentration of an endogenous substance of interest (e.g., Trp and Kyn) in the bilayer and in water. While concertation of a substance in the aqueous phase can easily be determined by a suitable analytical procedure (e.g., HPLC, LC-MS etc.), determination of the concertation in the bilayer requires two assumptions. First assumption is based on the fact that GMO has very low solubility in water around 10⁻⁶ M¹⁶ with an overall HLB of 3.8 implying that there is no free GMO existing in the aqueous phase (i.e., all GMO makes up the lipid bilayer). The second assumption is that the concentration of an endogenous substance in the water channels of the cubic phase is the same as in the water bulk phase, which is based on the equilibrium between chemical potentials of endogenous substances in water channels and in the bulk phase. Thus, the partition coefficient may be calculated by rewriting the Eq. 2 as follows

$${K}_{b 1 / w} = ( {V}_{w} ( {[ X ]}_{0 w} - {[ X ]}_{w} ) {/ [ X ]}_{w} - {V}_{cube} ) / {V}_{b 1} \qquad Eq . 3$$

where V_{w} is the volume of a water solution containing endogenous substance X added to the cubic phase, [X]_{0w} is the initial concentration of an endogenous substance, [X]_{w} is the concentration of endogenous substance in the water phase after equilibration, V_{bl} is the volume of GMO, V_{cube} is the volume of water that was added to GMO to form a cubic phase.

### HPLC-UV Analysis

The quantification of Trp and Kyn was performed by HPLC-UV system (Agilent 1100 Series, Germany). The chromatographic separation of Trp and Kyn was carried out on 250 mm×4.6 mm Kromasil C18 column with particle size of 5 μm (AkzoNobel, Bellefonte, USA). Endogenous substances were separated by gradient elution using mobile phase A consisting of 10 mM NaH₂PO₄ (pH 2.8) and mobile phase B consisting of 100% MeOH at 0.9 mL/min flow rate and 40° C. column temperature. The gradient profile was as follows: mobile phase B was kept at 25% for 7 minutes, then phase B was gradually increased to 95% over 4 minutes and kept at 95% for 4 minutes, after which phase B was decreased to 25% over 0.1 minute and kept at 25% for the final 1.9 minutes. The total run time was 17 minutes and injection volume was set to 20 μL. Detection of Trp and Kyn was performed at their UV absorbance maxima, at 280 nm and 360 nm, respectively. Stock solutions of 10 mM of Trp and Kyn for calibration curve were prepared in Milli-Q water and kept in the freezer (−20° C.) for no longer than one day after preparation. Calibration standards for calibration curve were analyzed in the range 0.78 μM to 100 μM (R²>0.999) the same day as the experimental samples. The amount of endogenous substances was determined by manual integration of the corresponding peaks using OpenLAB software (Lab Advisor Basic Software, Agilent, Germany). The concentrations of Trp and Kyn in the unknown samples were determined using the calibration curve obtained from standards solutions.

### Skin Preparation

Pig skin, purchased from a local abattoir, was gently cleaned post-sacrifice under cold running water. The skin from abdomen or from the inside of the outer ear was used as in vitro skin model. The hair was trimmed and the skin was dermatomed to a final thickness of 750 μm (Dermatome, Integra LifeSciences, Plainsboro, NJ, USA). Skin samples were then wrapped individually in Parafilm™ and kept at −20° C. for not more than three months until use. Prior to extraction experiments, skin was cut into 4 cm² membranes while still being frozen. Prepared skin membranes were then left at ambient environment for 30 minutes to thaw and obtain room temperature. The integrity of each skin membrane was visually inspected against the light in order to ensure that there were no visible damages (holes). Hairs were then further trimmed with scissors and skin was gently rinsed under cold running water.

### Preparation of Liquid Crystalline Cubic Phase

The samples for reverse iontophoretic extraction experiments consisting of a liquid crystalline cubic phase were prepared by weighing 0.1 g of glycerol monooleate (GMO) in a 1.8 mL glass vials. GMO was then melted at 45° C. by immersing the vials in the water bath and centrifuged at 1000 g for 5 minutes. The vials were then placed in the freezer (−20° C.) for 30 minutes in order for GMO to crystalize. After crystallization vials were taken out of the freezer and 0.5 g of Milli-Q water was added to each vial, ensuring the excess of added water. Clear and highly viscous liquid crystalline cubic phase was formed within 2 days. Prior to use, each vial was examined between cross polarizers for any sign of anisotropy, which would imply that sample had not yet reached equilibrium.

### Preparation of Electrodes

The Ag/AgCl electrode couple were preferred over platinum because it avoids the sharp decrease in the pH of the solution as their electrochemistry occurs at voltages considerably lower than those required for electrolysis of water. The electrodes were prepared by making a small loop at one of the ends of the silver wire and dipping it into the molten silver chloride in order to coat the loop with AgCl. After cooling, anodal electrodes were prepared by conditioning AgCl coated loops overnight against a platinum wire anode (0.2 mm in diameter, Sigma), at 0.3 mA with 50 mM NaCl as an electrolyte solution resulting in the formation of a layer of silver on the outer surface of the electrodes.

### Reverse Iontophoretic Extraction of Trp and Kyn

In iontophoresis the total iontophoretic flux of a substance is a sum of the contributions of three factors: electromigration, electroosmosis and passive diffusion. However, the passive diffusion is usually neglected since its contribution is much smaller (for the intact skin barrier) compared to the other two factors53. Therefore, the total iontophoretic flux may be expresses as

$${J}_{total} = {J}_{electromigration} + {J}_{electroosmosis} = \frac{{z}_{p} {u}_{p} {c}_{p} {I}_{D}}{F \cdot \sum {z}_{i} {u}_{i} {c}_{i}} + v \cdot {c}_{p} \qquad Eq . 4$$

where zₚ, uₚ, cₚ are the charge, mobility and concentration of the permeant, I_{D} is the current density (=I/A), F is the Faraday's constant and summation includes all the species in solution that can carry the charge, v is the solvent velocity. Electromigration is a direct cause of current application, which leads to a formation of an electric field across the skin. Interaction of small charged species with the established electric field results in their ordered movement towards electrode compartments of opposite charge to maintain the electroneutrality. Electroosmosis originates from the fact that skin has a net negative charge at physiological pH (skin pI 4-4.5) resulting in the skin's permselectivity to cations. Movement of cations in the electrical field established in the skin causes the convective solvent flow in the anode-to-cathode direction, which carriers along with it uncharged polar molecules and enhances the transport of cations, while impedes the transport of anions. Since most amino acids are in their zwitterionic state, electroosmosis is the primary mechanism of their extraction from the skin. Reverse iontophoretic experiments were performed in horizontal side-by-side cells consisting of two receptor compartments, anodal and cathodal (2 mL each), and a subdermal ‘donor’ compartment (3 mL). Two skin membranes were mounted per each cell in the way that SC was facing anode and cathode receptor compartments whereas the epidermal side was facing the donor compartment. The area available for transport was 0.785 cm² (FIG. 17 F). In all experiments, the donor compartment was filled with PBS (130.9 mM NaCl, 5.1 mM Na₂HPO₄ and 1.5 mM KH₂PO₄, pH 7.4) containing 1 mM of Trp and 1 mM of Kyn (subdermal Trp/Kyn=1). The background electrolyte solution in the receptor compartments (anodal and cathodal) was 10 mM HEPES, 60 mM NaCl buffered at one of the following pH values: 4.0, 7.4 and 9.0. Receptor solutions with different pH were used in order to investigate the effect of pH on the extraction efficiency. All solutions were degassed by sonication prior to extraction experiments. Extraction experiments with a cubic phase were performed in a similar manner using the same horizontal side-by-side cell setup. In order to ensure that the same amount of cubic phase was applied at each extraction site, custom made gel holders with the same area available for transport as side-by-side cells were prepared. The total volume of the cubic phase which fit in the gel holder was around 100 μL. After the assembly of side-by-side cells, anode (Ag) and cathode (Ag/AgCl) electrodes were inserted in each respective receptor compartment. Magnetic bars were used to achieve constant stirring throughout the experiment. Reverse iontophoretic extraction of Trp and Kyn was performed at room temperature (21.5±0.7° C.) by passing a constant current (−0.4 mA/cm²) for six hours. The current was generated by a commercial power supply (Yokogawa 7651 Programmable DC source, Woodburn Green, UK).

In extraction experiments preformed without a cubic phase, sampling was performed every hour for six hours by withdrawing 1 mL from the receptor compartments and replacing it with 1 mL of neat buffer solution. After the final sampling the current was stopped and the donor compartment was emptied. The post iontophoretic passive extraction from the skin membranes was allowed to continue for another 18 hours in order to investigate the effect of electroosmosis, since the skin membrane was the only source of endogenous substance after removal of donor solution. In total 7 samplings were performed over 24 hours i.e., 6 during the current passage and 1 from the post iontophoretic passive extraction. In case of reverse iontophoretic extraction into the cubic phase, the sampling was performed in the same way i.e., every hour for 6 hours 1 mL of receptor solution was replaced with a 1 mL of a neat buffer. However, in contrast to previous procedure, the experiment was terminated after 6 hours of current passage. The cubic phase was collected into 1.7 mL Eppendorf tube ensuring that as little residue as possible was left on the skin surface. The extraction of endogenous substances from the cubic phase was performed as follows: 1 mL of Milli-Q water was added to the cubic phase and left on shaker for 1 hour. After that, each Eppendorf tube was vortex for 1 minute and the final aqueous solution was collected with a syringe. Small part of a cubic phase was taken for SAXD measurements in order to investigate any potential changes in the phase after reverse iontophoretic experiment.

### Passive Diffusion Extraction of Trp and Kyn

In order to determine if reverse iontophoresis can enhance the extraction of Trp compared to their passive diffusion extraction, similar experiments were conducted without the current passage. These passive diffusion experiments were conducted using the same horizontal side-by-side cell setup in parallel to reverse iontophoresis experiments in order to ensure identical conditions. In line with reverse iontophoretic experiment, after the 6-hour sampling, subdermal compartment was emptied and experiment was allowed to continue for another 18 hours. Passive diffusion experiments were repeated four times (n=4).

### Control Experiment

Control reverse iontophoretic extraction experiments were conducted in order to estimate the levels of endogenous Trp and, potentially Kyn. It is known that Trp is present in the SC as a part of natural moisturizing factor (NMF), which primarily consists of free amino acids and their derivatives55,56. These experiments were performed using the identical experimental setup with a neat PBS buffer (pH 7.4) in the donor compartment (i.e., no Trp or Kyn being present) and 10 mM HEPES with 60 mM NaCl as background electrolyte (pH 7.4). Sampling was performed once an hour for 6 hours by withdrawing 1 mL of receptor solution and replacing it with a 1 mL of fresh HEPES buffer).

### Analytical Method

All samples collected from the reverse iontophoresis and from passive diffusion experiments were filtered with a 0.2 μm syringe filters (Minisart™ RC-15, Sartorius, UK) and kept in the freezer (˜20° C.) for no longer than one day after collection. Prior to HPLC-UV analysis, samples were thawed and vortexed. The quantification of Trp and Kyn was performed by HPLC-UV system (Shimadzu LC-2010 A HT system, Buckinghamshire, UK). The chromatographic separation of Trp and Kyn was carried out on 250 mm×4.6 mm C18 HiQ Sil column with particle size of 3 μm (Kromatech, Dunmow, UK). Endogenous substances were separated by gradient elution using mobile phase A consisting of 10 mM NaH2PO4 (pH 2.8) and mobile phase B consisting of 100% MeOH at 0.9 mL/min flow rate and 40° C. column temperature. The gradient profile was adopted from previous work and modified as follows: 0.0-7.0 min mobile phase B was kept at 25%, 7.0-11.0 min phase B was gradually increased to 95%, 11.0-15.0 min phase B was kept at 95%, 15.0-15.1 min phase B was decreased to 25% and kept for 1.9 min. The total run time was 17 min and injection volume was set to 20 μL. Detection of Trp and Kyn was performed at their UV absorbance maxima, at 280 nm and 360 nm, respectively. Stock solutions of 20 mM of Trp and Kyn for calibration curve were prepared in Milli-Q water and kept in the freezer (−20° C.) for no longer than one day after preparation. Calibration standards for calibration curve were analysed in triplicates (0.78 μM to 100 μM, R²>0.999) the same day as the experimental samples. The amount of endogenous substances was determined by manual integration of the corresponding peaks using LabSolution software (Shimadzu, Kyoto, Japan). The concentrations of Trp and Kyn in the unknown samples were determined using the calibration curve obtained from standards solutions.

### Small Angle X-Ray Diffraction

Small angle X-ray diffraction (SAXD) was used for phase characterization. All SAXD measurements were carried out on Xeuss 3.0 SAXS/WAXS laboratory-based instrument (Xenocs, France) at Malmö University (Malmö, Sweden). In this instrument, the X-ray beam is generated by Cu Kα radiation source (λ=1.541 Å). All samples were measured in an ambient environment at 25° C., with a temperature-controlled Peltier gel-holder stage using an O-ring as a spacer between two Kapton films (DuPont™ Kapton®, 0.013 mm thickness, Goodfellow, England). The diffraction data was collected by Pilatus3 R 300K hybrid photon counting detector with a sample-to-detector distance (STDD) of 800 mm. This STDD covers the q-range 0.0004≤q (Å-1)≤0.36, where q is the scattering vector defined as

$$\vert q \vert = q = \frac{4 π}{λ} sin ( \frac{θ}{2} )$$

and θ is the scattering angle. One-dimensional (1 D) data was obtained by azimuthal averaging of 2D-diffraction pattern and scattering intensity was corrected for background scattering and normalised to direct beam. The exposure time was 20 minutes for each sample. The lattice parameter, a, which is a measure of a smallest repeat distance in the unit cell, was calculated in order to determine potential changes in the cubic phase. For cubic phases, the lattice parameter is defined as

$${d}_{h k l} = \frac{{a}_{c u b}}{\sqrt{{h}^{2} + {k}^{2} + {l}^{2}}} ,$$

where h, k and l are Miller indices of Bragg's peak and dhkl is the repeat distance between atomic planes, which comes from Bragg's law nλ=2dₕₖₗ sin θ, n is the reflection order.

### Data Analysis and Statistics

Anodal, cathodal and passive fluxes of Trp and Kyn were directly calculated from the amount (mol) extracted at each sampling interval and normalized for the surface area available for extraction (0.785 cm²) and duration of the interval (in hours). The 6-hour flux values were used for the analysis of transport direction as well for comparison between reverse iontophoretic and passive extraction efficiency. The physiochemical properties of both endogenous substances were estimated. In order to compare results, the enhancement ratio (ER) was determined after 6 hours as

$$ER = \frac{cumulative amount extracted with current}{cumulative extracted by passive diffusion} \qquad Eq . 5$$

When appropriate, the resulting data were represented as mean±standard error of the mean (SEM). All statistical analysis was performed using R studio (Version 1.3.1093, R Foundation for Statistical Computing, Vienna, Austria). The level of statistical significance was fixed at α≤0.05.

1. A matrix or a patch comprising said matrix for non-invasive sampling of at least one endogenous substance on a skin surface of an individual, wherein the matrix comprises at least one amphiphile, wherein the amphiphile, alone or in combination with at least one structurally related amphiphile, forms a non-lamellar liquid crystalline phase together with an aqueous polar solvent mixture, said matrix comprising a water activity of at least 0.85 in a temperature range of 20-40° C., wherein the matrix is configured to extract said at least one endogenous substance on the skin surface of the individual.

2. The matrix or patch according to claim 1, wherein the non-lamellar liquid crystalline phase is selected from the group consisting of: cubic phase, hexagonal phase, micellar, sponge phase and any intermediate between these or mixtures thereof.

3. The matrix or patch according to claim 1, wherein the amphiphile is selected from the group consisting of: natural lipids, synthetic lipids, ionic lipids and surfactants, preferably the ionic lipids are selected from the group consisting of: anionic lipids, cationic lipids and zwitterionic lipids, preferably the cationic lipids are selected from the group consisting of: dioleoyl-3-trimethylammonium propane, and dipalmitoyl-3-trimethylammonium propane, dipalmityl-3-trimethylammonium propane, distearyl-3-trimethylammonium propane and dielaidyl-3-trimethylammonium propane or mixtures thereof, preferably the cationic lipid is dioleoyl-3-trimethylammonium propane.

4. The matrix or patch according to claim 1, wherein the amphiphile is selected from the group consisting of: glyceryl monooleate, glyceryl monoelaidate, glyceryl monolinoleate, glyceryl dioleate, dioleyl phosphatidylglycerol, distearyl phosphatidylglycerol, dioleyl phosphatidyl ethanolamine, dioleyl phosphatidylcholine and phytantriol or mixtures thereof.

5. The matrix or patch according to claim 1, wherein the matrix further comprises at least one additive selected from the group consisting of: humectant, drug, bioactive agent, irritant and allergen.

6. The matrix or patch according to claim 1, wherein the aqueous polar solvent mixture comprises water or water in combination with a polar co-solvent, such as ethanol, glycerol or isopropyl alcohol.

7. (canceled)

8. A method of using the matrix or patch according to claim 1, the method comprising:
i. placing the matrix or the patch against an area of the skin surface of an individual;
ii. extracting at least one endogenous substance on the area of the skin surface of the individual; and
iii. determining the presence of said at least one endogenous substance.

9. The method according to claim 8, wherein the at least one endogenous substance is extracted with other endogenous substances or metabolites thereof and the determining step iii comprises estimating a ratio between two of the extracted endogenous substances or metabolites.

10. The method according to claim 8, further comprising:
delivering a drug or a bioactive agent prior to and/or simultaneously to step ii from the matrix, to trigger a response that reflects the presence of the at least one extracted endogenous substance.

11. The method according to claim 8, further comprising:
delivering an irritant or an allergen prior to and/or simultaneously to step ii from the matrix, to estimate if said irritant or allergen trigger a response in the skin of the individual, and thus to distinguish between an irritant, allergic or toxic effect of said endogenous substance on the individual.

12. The method according to claim 8, wherein said at least one endogenous substance is a low molecular weight substance with a molecular weight of up to 1000 Da, typically up to 500 Da.

13.-15. (canceled)

16. The method according to claim 8, wherein extracting at least one endogenous substance on the area of the skin surface of the individual comprises the use of reverse iontophoresis.

17. The method according to claim 8, wherein said at least one endogenous substance is associated with inflammatory diseases selected from the group consisting of: seborrheic dermatitis, rosacea, lupus, psoriasis, eczema, ichtyosis, skin cancer, diabetes and inflamatory bowel disease.
